(19) United States
(12) Patent Application Publication
(10) Pub. No.: US 2025/0019375 A1
(43) Pub. Date: Jan. 16, 2025

(54) BENZOTHIAZOLE DYES

(71) Applicant: Detect, Inc., Guilford, CT (US)

(72) Inventors: David N. Kamber, Guilford, CT (US); Roger R. Nani, Madison, CT (US); William A. Hansen, Clinton, CT (US)

(21) Appl. No.: 18/758,670

(22) Filed: Jun. 28, 2024

Related U.S. Application Data
(60) Provisional application No. 63/511,610, filed on Jun. 30, 2023.

(51) Int. Cl. C07D 417/06; C07D 413/06; C07D 417/14; C12Q 1/689; C12Q 1/70; G01N 21/64

(52) U.S. Cl. CPC C07D 417/06; C07D 413/06; C07D 417/14; C12Q 1/689; C12Q 1/701; G01N 21/6428; C12Q 2600/158; G01N 2021/6439

(57) ABSTRACT

Provided herein are compounds having aqueous solubility, stability, and fluorescence, as well as methods of detecting the presence or absence of an analyte in a test sample using said compounds.

### CROSS-REFERENCE TO RELATED APPLICATIONS

The present application claims the benefit of and priority under 35 U.S.C. § 119(e) to U.S. Provisional Application No. 63/511,610 filed Jun. 30, 2023, titled BENZOTHIAZOLE DYES, the contents of which are incorporated herein by reference in their entirety.

### BACKGROUND

Dyes are commonly used in the biotechnological and biomedical research fields for the detection of molecules of interest, such as nucleic acids. The detection and quantification of nucleic acids, particularly DNA, using fluorescent dyes in biological test samples is an important tool in the diagnosis of various genetic or pathological conditions. As more sophisticated diagnostic and testing platforms are developed, there is an increased need for dye compounds having properties amenable to the new mechanical and chemical requirements of the platforms.

### SUMMARY OF THE INVENTION

In one aspect, the present disclosure provides a compound of Formula (I):

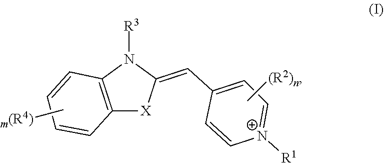

or a salt thereof, wherein:
n is 1-4;
m is 0-4;
X is oxygen or sulfur;
R¹ is hydrogen, substituted or unsubstituted alkyl, substituted or unsubstituted heteroalkyl, or substituted or unsubstituted heteroaryl;
R² is, independently for each occurrence, halo, —OR⁵, —SR⁵, —N(R⁵)₂, substituted or unsubstituted alkyl, substituted or unsubstituted heteroalkyl, substituted or unsubstituted carbocyclyl, substituted or unsubstituted heterocyclyl, substituted or unsubstituted aryl, or substituted or unsubstituted heteroaryl; wherein two adjacent R² groups may combine to form a fused substituted or unsubstituted aryl, a fused substituted or unsubstituted heteroaryl, a fused substituted or unsubstituted carbocyclyl, or a fused substituted or unsubstituted heterocyclyl;
R³ is substituted or unsubstituted alkyl;
R⁴ is, independently for each occurrence, halo, —NO₂, —OR⁶, —SR⁶, —N(R⁶)₂, substituted or unsubstituted alkyl, substituted or unsubstituted heteroalkyl, substituted or unsubstituted carbocyclyl, substituted or unsubstituted heterocyclyl, substituted or unsubstituted aryl, or substituted or unsubstituted heteroaryl;
R⁵ is, independently for each occurrence, hydrogen, substituted or unsubstituted alkyl, nitrogen protecting group when attached to a nitrogen, oxygen protecting group when attached to an oxygen, or a sulfur protecting group when attached to a sulfur, or when R² is —N(R⁵)₂, two instances of R⁵ may be joined to form a substituted or unsubstituted heterocyclyl or a substituted or unsubstituted heteroaryl; and
R⁶ is, independently for each occurrence, hydrogen, substituted or unsubstituted alkyl, nitrogen protecting group when attached to a nitrogen, oxygen protecting group when attached to an oxygen, or a sulfur protecting group when attached to a sulfur.

In another aspect, the present disclosure provides a composition comprising a compound described herein, or a salt thereof.

In another aspect, the present disclosure provides a method of detecting the presence or absence of an analyte in a test sample, comprising contacting the test sample with a compound of the present disclosure in the presence of an energy (E1) and detecting emission of an energy (E2) from the sample.

In another aspect, the present disclosure provides a kit comprising: a compound disclosed herein; or a composition of the present disclosure; and instructions for use.

### BRIEF DESCRIPTION OF THE DRAWINGS

The accompanying drawings, which constitute a part of this specification, illustrate several embodiments of the invention and together with the description, provide non-limiting examples of the invention.

FIG. 1A shows time to positive (Tp) data for Chlamydia trachomatis (RS2340-16) qLAMP assay with titration of dyes (4.0 μM, 8.0 μM, 10.0 μM, 16.0 μM), left=clean positive control, right=clean negative control. FIG. 1B shows Tp data for Neisseria gonorrhoeae trachomatis (WX60) qLAMP assay with titration of dyes (4.0 μM, 8.0 μM, 10.0 μM, 16.0 μM), left=clean positive control, right=clean negative control.

FIG. 2 shows Tp data for Neisseria gonorrhoeae trachomatis (AQ10) qLAMP assay with titration of dyes (4.0 μM, 8.0 μM, 10.0 μM, 16.0 μM), left=clean positive control, right=clean negative control.

FIG. 3A shows time to positive (RES) results for qLAMP assays using Flub, H1N1, SC2, and NG2 primer sets with compound 2 at varying concentrations (NTC=non-template control). FIG. 3B shows Tp (RES) results for qLAMP assays using Flub, H1N1, SC2, and NG2 primer sets with compound 1 at varying concentrations (NTC=non-template control).

FIG. 4 shows RES data for Neisseria gonorrhoeae trachomatis (AQ10) qLAMP assay with compound 3 at varying temperatures.

FIG. 5 shows stability data for compound 3 under accelerated stability conditions (40° C., lyophilized samples) over 1-3 months.

FIG. 6A-F show absorption/emission spectra of dyes with double stranded DNA (dsDNA). FIG. 6A shows a Stokes shift of 50 nm (λ_{abs}=448 nm and λₑₘ=498 nm) for compound 3. FIG. 6B shows a Stokes shift of 45 nm (λ_{abs}=448 nm and λₑₘ=493 nm) for compound 2. FIG. 6C shows a Stokes shift of 30 nm (λ_{abs}=469 nm and λₑₘ=499 nm) for compound 9. FIG. 6D shows a Stokes shift of 38 nm (λ_{abs}=442 nm and λₑₘ=480 nm) for compound 8. FIG. 6E shows a Stokes shift or 38 nm (λ_{abs}=443 nm and λₑₘ=481 nm) for compound 10. FIG. 6F shows a Stokes shift of 43 nm (λ_{abs}=510 nm and λₑₘ=553 nm) for compound 11.

FIG. 7 shows table of compound 3 characteristics compared to SYBR Gold and SYBR Safe, two commercially available nucleic acid stains.

FIG. 8 shows agarose gel staining with compound 3 and SYBR Gold in green, blue, and red fluorescence channels.

FIG. 9 shows the dynamic range of compound 3 compared to SYTO 9 with positive and negative controls.

FIG. 10 shows percent (%) lyophilized dye remaining for SYTO 9 (2 months at 25° C.) and compound 3 (25 days at 40° C.).

FIGS. 11A-B show the quantification of DNA concentration using compound 3. FIG. 11A shows a calibration curve of DNA with compound 3 measuring emission intensity at 498 nm. FIG. 11B shows the fluorescence emission data for compound 3 with and without DNA.

### DEFINITIONS

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The present disclosure additionally encompasses compounds as individual isomers substantially free of other isomers, and alternatively, as mixtures of various isomers.

In a formula, the bond [symbol] is a single bond, the dashed line [symbol] is a single bond or absent, and the bond [symbol] or [symbol] is a single or double bond.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

The term “isotopes” refers to variants of a particular chemical element such that, while all isotopes of a given element share the same number of protons in each atom of the element, those isotopes differ in the number of neutrons. The term “radioactivity” or “radioactive decay” refers to the process by which a nucleus of an unstable isotope (e.g., ¹⁸F) loses energy by emitting particles or rays (e.g., alpha particles, beta particles, and gamma rays) of ionizing radiation. Such an unstable isotope or a material including the unstable isotope is referred to as “radioactive.” The Curie (Ci) is a non-SI (non-International System of Units) unit of radioactivity and is defined as 1 Ci=3.7×10¹⁰ decays per second. The term “specific activity” refers to the unit radioactivity of a material (e.g., a compound of disclosed herein, or a salt, tautomer, stereoisomer, or isotopically labeled derivative (e.g., ¹⁸F labeled derivative) thereof). In certain embodiments, the term “specific activity” refers to the radioactivity of a material per micromole (mol) of the material.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “heteroatom” refers to an atom that is not hydrogen or carbon. In certain embodiments, the heteroatom is nitrogen. In certain embodiments, the heteroatom is oxygen. In certain embodiments, the heteroatom is sulfur.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

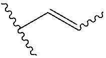

) may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like.

Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁, cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 □ electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
each instance of R^{cc} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂—CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxy,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “alcohol” us herein, refers to an optionally substituted alkyl group, as defined herein, appended to a hydroxyl group. Representative examples of alcohol groups include but are not limited to, methanol, ethanol, n-propanol, isopropanol, n-butanol, isobutanol, and t-butanol.

The term “alkoxy” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂—CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄1⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

These and other exemplary substituents are described in more detail in the Detailed Description, Examples, and Claims. The present disclosure is not limited in any manner by the above exemplary listing of substituents.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of the present disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “about X,” where X is a number or percentage, refers to a number or percentage that is between 99.5% and 100.5%, between 99% and 101%, between 98% and 102%, between 97% and 103%, between 96% and 104%, between 95% and 105%, between 92% and 108%, or between 90% and 110%, inclusive, of X.

The terms “composition” and “formulation” are used interchangeably.

The terms “polynucleotide”, “nucleotide sequence”, “nucleic acid”, “nucleic acid molecule”, “nucleic acid sequence”, “nucleotide”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonuculeotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA.

Polynucleotides described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Any type of plasmid, cosmid, yeast artificial chromosome, or viral vector can be used to prepare the recombinant DNA construct that can be introduced directly into the tissue site.

The polynucleotides may be flanked by natural regulatory (expression control) sequences or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications, such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, isotopes (e.g., radioactive isotopes), biotin, and the like.

The term “biological sample” and “test sample” are interchangeable as used herein and refer to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “crude sample matrix” or “sample matrix” as used herein represents a sample which is collected from a source (i.e., a mammal, or tissue culture) which has not undergone purification. In some embodiments, a crude sample matrix is a nasal swab, vaginal swab, anal swab, mucus, saliva, sputum, urine, blood, or cell scraping sample. In some embodiments, a crude sample matrix is a nasal swab. In some embodiments, a crude sample matrix is blood. In some embodiments, a crude sample matrix is urine. In some embodiments, a crude sample matrix is blood. In some embodiments, a crude sample matrix is a biological sample.

The term “clean negative” refers to a sample which does not comprise the crude sample matrix.

As used herein “RES” stands for results and is interchangeable with “time to positive” or “Tp,” and is in units of time (i.e., minutes).

As used herein, the term “stable” means that more than 95% of compound remains at various time points in different media (e.g., aqueous, lyophilized).

### DETAILED DESCRIPTION

The present disclosure relates to stable benzothiazole dyes that may be useful in applications such as isothermal nucleic acid amplification tests (NAAT).

Real-time monitoring of NAAT in clinical samples is valuable in distinguishing false positives and negatives. Fluorescence-based monitoring offers several advantages over other readout strategies, especially binary endpoint analyses (e.g., color change, byproduct precipitation, LFA, etc.).

Fluorescence readout strategies are highly sensitive and exhibit a wide dynamic range (signal to background) which can be monitored and analyzed in several ways in real time. For example, different aspects of the sigmoidal shape of a LAMP amplification curve, i.e., amplitude, slope, inflection points, first and second derivatives, etc., offer more information on the occurring amplification to aid more selective distinguishing of positive and negative samples. Utilizing a fluorogenic dye that is stable to hydrolysis, water soluble, and non-inhibitory to the speed of the NAAT is critical to manufacturing affordable, rapid (<15 min from sampling to result) and shelf stable NAAT consumable. Furthermore, it is critical to develop a fluorogenic dye with tunable optical properties to match with the inexpensive excitation sources and filters of <$15 test.

Disclosed herein are pyridyl-benzothiazole dyes found to provide a modifiable core structure that, after evaluation of multiple analogues, were shown to meet the requirements of a fluorogenic dye for an inexpensive NAAT test.

The use of pyridyl-benzothiazole dyes allows for optimal spectral overlap with the excitation source of the device (450 nm), which is necessary because most commercially available dyes for NAAT tests do not provide a sufficient output signal due to non-ideal overlap of their absorbance with the LED light source. The core structure of the pyridyl-benzothiazole dyes has allowed for versions with large Stokes shifts to allow a substantial portion of the output fluorescence to pass through the filter covering the photodiode detection component of the device.

The dyes are minimally hydrophobic which result in water solubility critical for formulation and lyophilization processes. The structural features of the pyridyl-benzothiazole dyes (low logP, single cationic charge) allow for the desired detection of target amplification products while minimizing inhibition of the isothermal amplification process which is critical for rapid tests.

The use of the benzothiazole moiety results in optimal spectral overlap with the LED and is more hydrolytically stable than the related benzoxazole series of dyes, which is important for shelf life of NAAT tests.

The benzothiazole dye compounds have improved aqueous solubility, stability, and large Stokes shift >40 nm (50 nm for compound 3 with dsDNA), representing improvements over benzoxazole based dyes. The analyte is a nucleic acid, double-stranded DNA. In the presence of dsDNA, the dyes emit a useful fluorescence signal upon irradiation and have been shown to be tolerant of up to 1% blood in LAMP based assays, providing a signal that is distinguished from background. In other examples, the nucleic acid is of a virus or bacterium.

The dyes are amenable to formulation into lyobeads and remain stable relative to commercially available benzoxazole dyes. Benzothiazole dyes that contain positively charged linker (dyes with two positive charges overall) were found to be inhibitory to LAMP at certain concentrations (e.g., >2 μM). Less hydrophobic structure (less pi-surface area) would result in less inhibition of LAMP with certain targets and primer sets.

In one aspect, provided is a compound of Formula (I):

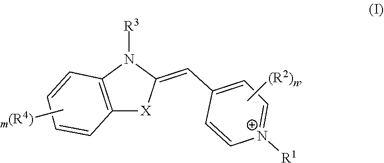

or a salt thereof, wherein:
n is 1-4;
m is 0-4;
X is oxygen or sulfur;
R¹ is hydrogen, substituted or unsubstituted alkyl, substituted or unsubstituted heteroalkyl, or substituted or unsubstituted heteroaryl;
R² is, independently for each occurrence, halo, —OR⁵, —SR⁵, —N(R⁵)₂, substituted or unsubstituted alkyl, substituted or unsubstituted heteroalkyl, substituted or unsubstituted carbocyclyl, substituted or unsubstituted heterocyclyl, substituted or unsubstituted aryl, or substituted or unsubstituted heteroaryl; wherein two adjacent R² groups may combine to form a fused substituted or unsubstituted aryl, a fused substituted or unsubstituted heteroaryl, a fused substituted or unsubstituted carbocyclyl, or a fused substituted or unsubstituted heterocyclyl;
R³ is substituted or unsubstituted alkyl;
R⁴ is, independently for each occurrence, halo, —NO₂, —OR⁶, —SR⁶, —N(R⁶)₂, substituted or unsubstituted alkyl, substituted or unsubstituted heteroalkyl, substituted or unsubstituted carbocyclyl, substituted or unsubstituted heterocyclyl, substituted or unsubstituted aryl, or substituted or unsubstituted heteroaryl;
R⁵ is, independently for each occurrence, hydrogen, substituted or unsubstituted alkyl, nitrogen protecting group when attached to a nitrogen, oxygen protecting group when attached to an oxygen, or a sulfur protecting group when attached to a sulfur, or when R² is —N(R⁵)₂, two instances of R⁵ may be joined to form a substituted or unsubstituted heterocyclyl or a substituted or unsubstituted heteroaryl; and
R⁶ is, independently for each occurrence, hydrogen, substituted or unsubstituted alkyl, nitrogen protecting group when attached to a nitrogen, oxygen protecting group when attached to an oxygen, or a sulfur protecting group when attached to a sulfur.

### X

As generally described herein, X is oxygen or sulfur. In some embodiments, X is sulfur. In some embodiments, X is oxygen.

### R¹

As generally described herein, R¹ is hydrogen, substituted or unsubstituted alkyl, substituted or unsubstituted heteroalkyl, or substituted or unsubstituted heteroaryl. In certain embodiments, R¹ is hydrogen, substituted or unsubstituted alkyl, or substituted or unsubstituted heteroalkyl. In certain embodiments, R¹ is hydrogen. In certain embodiments, R¹ is substituted or unsubstituted heteroalkyl.

In some embodiments, R¹ is not substituted or unsubstituted heteroaryl.

In certain embodiments, R¹ is substituted or unsubstituted alkyl. In some embodiments, R¹ is substituted or unsubstituted C₁₋₁₂ alkyl. In some embodiments, R¹ is substituted or unsubstituted C₁₋₆ alkyl. In certain embodiments, R¹ is substituted alkyl.

In some embodiments, R¹ is substituted or unsubstituted C₁₋₃ alkyl. In some embodiments, R¹ is unsubstituted C₁₋₃ alkyl. In some embodiments, R¹ is substituted C₁₋₃ alkyl.

In certain embodiments, R¹ is unsubstituted alkyl.

In some embodiments, R¹ is methyl. In some embodiments, R¹ is ethyl. In some embodiments, R¹ is propyl.

In certain embodiments, R¹ is alkyl substituted with an amine. In some embodiments, R¹ is substituted C₁₋₃ alkyl substituted with an amine.

In some embodiments, R¹ is alkyl substituted with a quaternary ammonium. In some embodiments, R¹ is substituted C₁₋₃ alkyl substituted with a quaternary ammonium. In some embodiments, R¹ is substituted C₁₋₃ alkyl substituted with trimethylammonium. In some embodiments, R¹ is —(CH₂)₃N⁺(Me)₃. In some embodiments, R¹ is —(CH₂)₃N⁺(H)₃.

In certain embodiments, X is sulfur and R¹ is hydrogen, substituted or unsubstituted alkyl, or substituted or unsubstituted heteroalkyl. In certain embodiments, X is oxygen and R¹ is hydrogen, substituted or unsubstituted alkyl, or substituted or unsubstituted heteroalkyl.

n

As generally described herein, n is 1-4. In certain embodiments, n is 1, 2, 3, or 4. In certain embodiments, n is 1 or 2. In certain embodiments, n is 1. In certain embodiments, n is 2. In certain embodiments, n is 3. In certain embodiments, n is 4.

In some embodiments, the compound of Formula (I) is of the Formula (I-a) or (I-b):

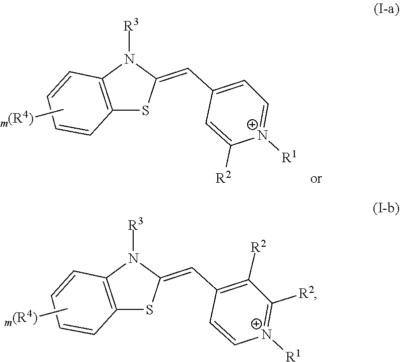

or a salt thereof.

In some embodiments, the compound of Formula (I) is of the Formula (I-a):

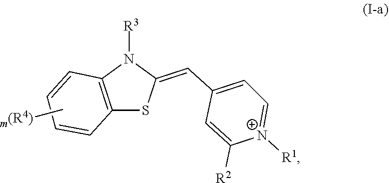

or a salt thereof. In certain embodiments, the compound of Formula (I) is of the Formula (I-a), or a salt thereof, wherein R¹ is hydrogen, substituted or unsubstituted alkyl, or substituted or unsubstituted heteroalkyl.

In some embodiments, the compound of Formula (I) is of the Formula (I-b):

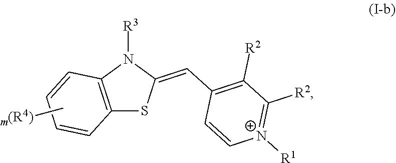

or a salt thereof. In certain embodiments, the compound of Formula (I) is of the Formula (I-b), or a salt thereof, wherein R¹ is hydrogen, substituted or unsubstituted alkyl, or substituted or unsubstituted heteroalkyl.

### R²

As generally described herein, R² is, independently for each occurrence, halo, —OR⁵, —SR⁵, —N(R⁵)₂, substituted or unsubstituted alkyl, substituted or unsubstituted heteroalkyl, substituted or unsubstituted carbocyclyl, substituted or unsubstituted heterocyclyl, substituted or unsubstituted aryl, or substituted or unsubstituted heteroaryl; wherein two adjacent R² groups may combine to form a fused substituted or unsubstituted aryl, a fused substituted or unsubstituted heteroaryl, a fused substituted or unsubstituted carbocyclyl, or a fused substituted or unsubstituted heterocyclyl.

In some embodiments, R² is, independently for each occurrence, halo. In some embodiments, R² is, independently for each occurrence, —OR⁵. In some embodiments, R² is, independently for each occurrence, —SR⁵. In some embodiments, R² is, independently for each occurrence, —N(R⁵)₂. In some embodiments, R² is, independently for each occurrence, substituted or unsubstituted alkyl. In some embodiments, R² is, independently for each occurrence, substituted or unsubstituted heteroalkyl. In some embodiments, R² is, independently for each occurrence, substituted or unsubstituted carbocyclyl. In some embodiments, R² is, independently for each occurrence, substituted or unsubstituted heterocyclyl. In some embodiments, R² is, independently for each occurrence, substituted or unsubstituted aryl. In some embodiments, R² is, independently for each occurrence, substituted or unsubstituted heteroaryl. In some embodiments, two adjacent R² groups combine to form a fused substituted or unsubstituted aryl, a fused substituted or unsubstituted heteroaryl, a fused substituted or unsubstituted carbocyclyl, or a fused substituted or unsubstituted heterocyclyl. In some embodiments, two adjacent R² groups combine to form a fused substituted or unsubstituted aryl.

In some embodiments, at least one R² is substituted or unsubstituted alkyl. In certain embodiments, at least one R² is substituted or unsubstituted heterocyclyl, substituted or unsubstituted aryl, substituted or unsubstituted heteroaryl, or —N(R⁵)₂. In certain embodiments, at least one R² is substituted or unsubstituted heterocyclyl. In certain embodiments, at least one R² is substituted or unsubstituted aryl. In certain embodiments, at least one R² is substituted or unsubstituted heteroaryl, or —N(R⁵)₂.

In some embodiments, two adjacent R² groups combine to form a fused substituted or unsubstituted aryl. In some embodiments, two adjacent R² groups combine to form a fused substituted or unsubstituted heteroaryl. In some embodiments, two adjacent R² groups combine to form a fused substituted or unsubstituted carbocyclyl. In some embodiments, two adjacent R² groups combine to form a fused substituted or unsubstituted heterocyclyl.

In certain embodiments, at least one R² is selected from:

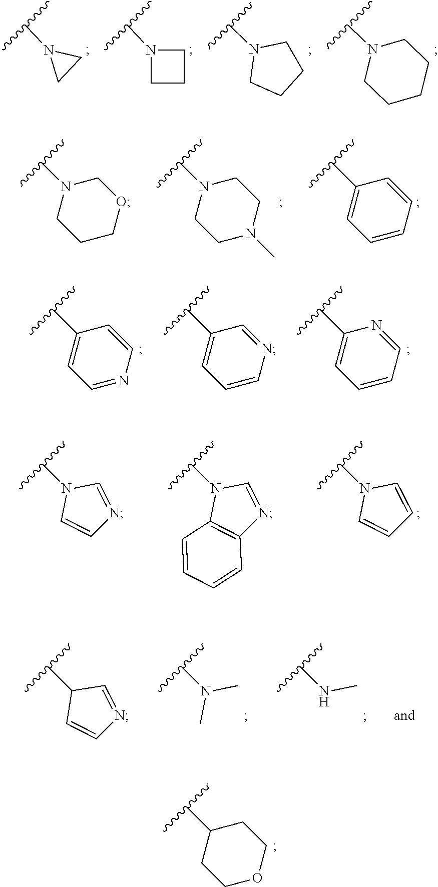

or two adjacent R² groups combine to form

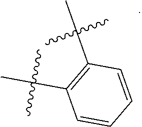 [SMILES: *c1ccccc1*]

In some embodiments, at least one R² is

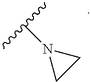 [SMILES: *N1CC1]

In some embodiments, at least one R² is

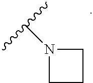 [SMILES: *N1CCC1]

In some embodiments, at least one R² is

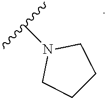 [SMILES: *N1CCCC1]

In some embodiments at least one R² is

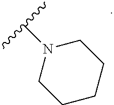 [SMILES: *N1CCCCC1]

In some embodiments, at least one R² is

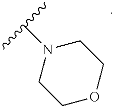 [SMILES: *N1CCOCC1]

In some embodiments, at least one R² is

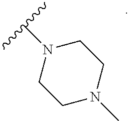 [SMILES: *N1CCN(C)CC1]

In some embodiments, at least one R² is

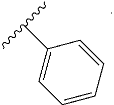 [SMILES: *c1ccccc1]

In some embodiments, at least one R² is

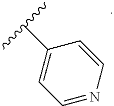 [SMILES: *c1ccncc1]

In some embodiments, at least one R² is

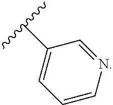 [SMILES: *c1cccnc1]

In some embodiments, at least one R² is

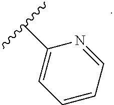 [SMILES: *c1ccccn1]

In some embodiments, at least one R² is

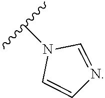 [SMILES: *n1ccnc1]

In some embodiments, at least one R² is

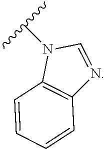 [SMILES: *n1cnc2ccccc21]

In some embodiments, at least one R² is

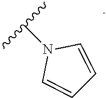 [SMILES: *n1cccc1]

In some embodiments, at least one R² is

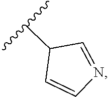 [SMILES: *C1C=CN=C1]

In some embodiments, at least one R² is

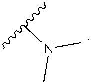 [SMILES: *N(C)C]

In some embodiments, at least one R² is

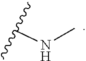 [SMILES: *NC]

In some embodiments, at least one R² is

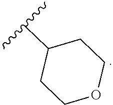 [SMILES: *C1CCOCC1]

In some embodiments, two adjacent R² groups combine to form

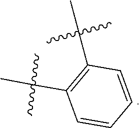 [SMILES: *c1ccccc1*]

In some embodiments, at least one R² is selected from:

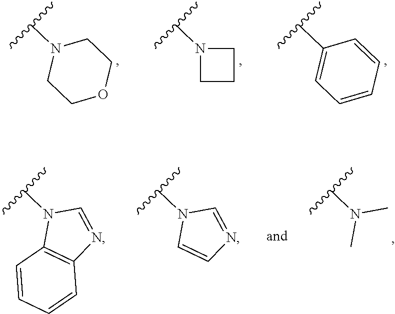

or two adjacent R² groups combine to form

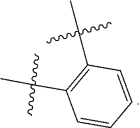 [SMILES: *c1ccccc1*]

In some embodiments, at least one R² is

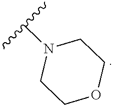 [SMILES: *N1CCOCC1]

In some embodiments, the compound of Formula (I) is of the Formula (I-a), or a salt thereof, wherein R¹ is hydrogen, substituted or unsubstituted alkyl, or substituted or unsubstituted heteroalkyl; and R² is selected from:

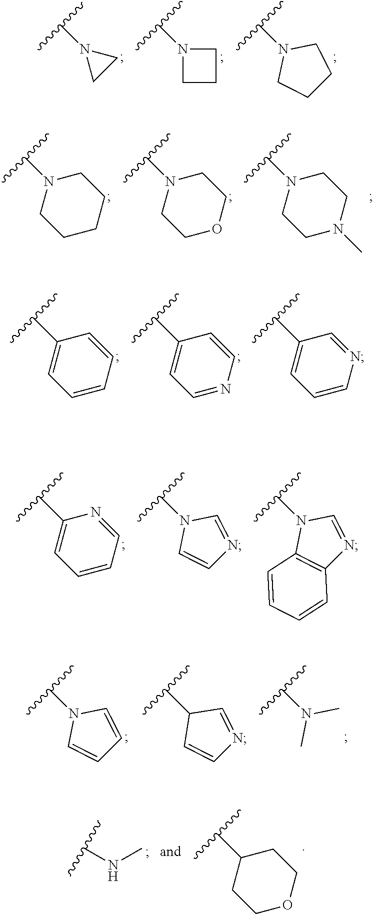

In some embodiments, the compound of Formula (I) is of the Formula (I-b), or a salt thereof, wherein R¹ is hydrogen, substituted or unsubstituted alkyl, or substituted or unsubstituted heteroalkyl; and at least one R² is selected from:

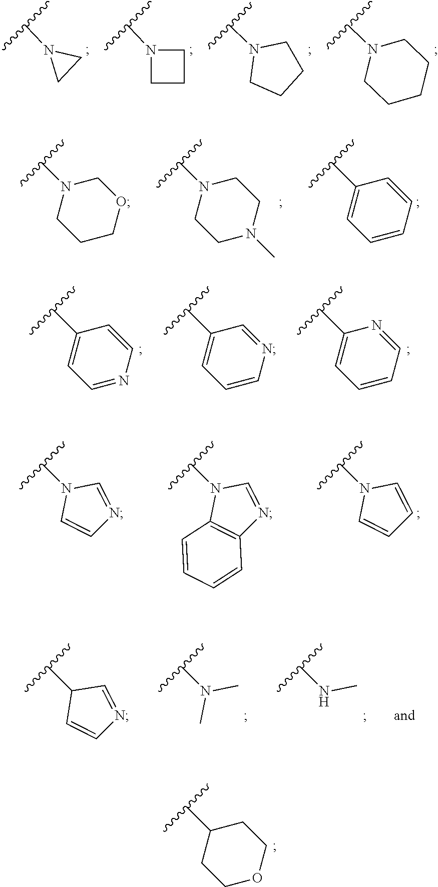

or two adjacent R² groups combine to form

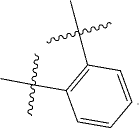 [SMILES: *c1ccccc1*]

### R³

As generally described herein, R³ is substituted or unsubstituted alkyl. In certain embodiments, R³ is unsubstituted alkyl. In certain embodiments, R³ is substituted alkyl. In certain embodiments, R³ is methyl.

In some embodiments, R³ is substituted or unsubstituted C₁₋₃ alkyl. In some embodiments, R³ is unsubstituted C₁₋₃ alkyl. In some embodiments, R³ is substituted C₁₋₃ alkyl.

In certain embodiments, R³ is alkyl substituted with an amine. In some embodiments, R³ is substituted C₁₋₃ alkyl substituted with an amine.

In some embodiments, R³ is alkyl substituted with a quaternary ammonium. In some embodiments, R³ is substituted C₁₋₃ alkyl substituted with a quaternary ammonium. In some embodiments, R³ is substituted C₁₋₃ alkyl substituted with trimethylammonium. In some embodiments, R³ is —(CH₂)₃N⁺(Me)₃. In some embodiments, R³ is —(CH₂)₃N⁺(H)₃.

In some embodiments, the compound of Formula (I) is of the Formula (I-a), or a salt thereof, wherein R¹ is hydrogen, substituted or unsubstituted alkyl, or substituted or unsubstituted heteroalkyl; R² is selected from

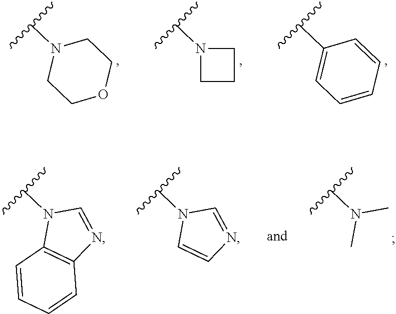

and R³ is unsubstituted alkyl.

In some embodiments, the compound of Formula (I) is of the Formula (I-b), or a salt thereof, wherein R¹ is hydrogen, substituted or unsubstituted alkyl, or substituted or unsubstituted heteroalkyl; at least one R² is selected from

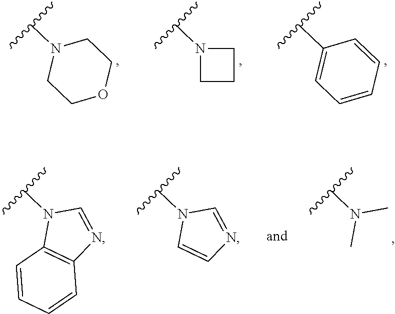

or two adjacent R groups combine to form

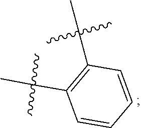 [SMILES: *c1ccccc1*]

and R³ is unsubstituted alkyl.

In some embodiments, the compound of Formula (I) is of Formula (I-c):

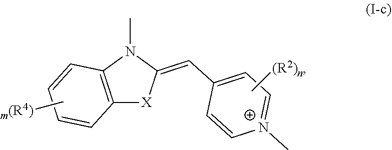

or a salt thereof. In some embodiments, the compound of Formula (I) is of Formula (I-c), or a salt thereof, wherein at least one R² is selected from

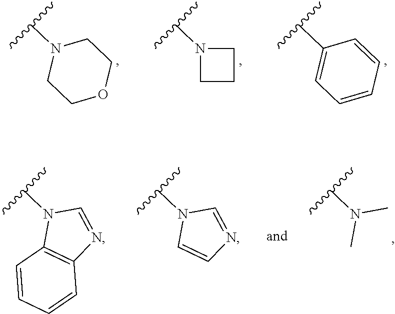

or two adjacent R² groups combine to form

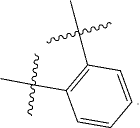 [SMILES: *c1ccccc1*]

In some embodiments, the compound of Formula (I) is of Formula (I-c-I) or Formula (I-c-II):

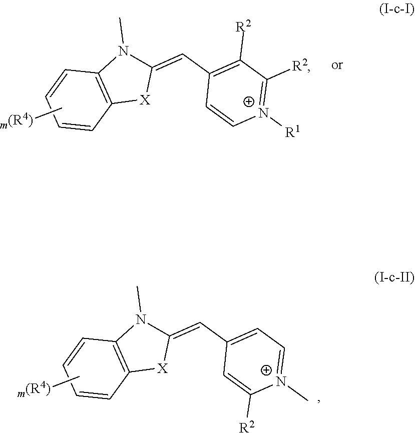

or a salt thereof. In some embodiments, the compound of Formula (I) is of Formula (I-c-I), or a salt thereof. In some embodiments, the compound of Formula (I) is of Formula (I-c-II), or a salt thereof.

In some embodiments, the compound of Formula (I) is of Formula (I-d):

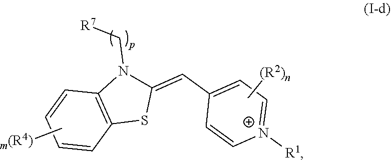

or a salt thereof, wherein:
p is 1-4; and
R⁷ is —CH₃ or a quaternary ammonium group.

In some embodiments, p is 3. In some embodiments, p is 1. In some embodiments, p is 2. In some embodiments, p is 4.

In certain embodiments, R⁷ is —CH₃. In some embodiments, R⁷ is —N⁺(Me)₃. In some embodiments, R⁷ is —N⁺(H)₃.

In some embodiments, the compound of Formula (I) is of Formula (I-d), or a salt thereof, wherein R¹ is hydrogen, substituted or unsubstituted alkyl, or substituted or unsubstituted heteroalkyl; and R² is selected from

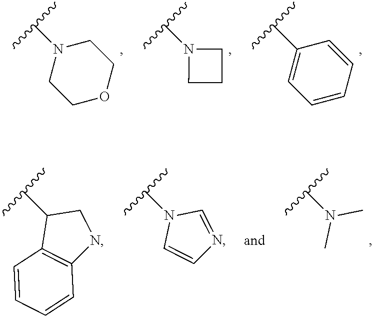

or two adjacent R² groups combine to form

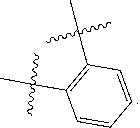 [SMILES: *c1ccccc1*]

In some embodiments, the compound of Formula (I) is of Formula (I-d-I) or (I-d-II):

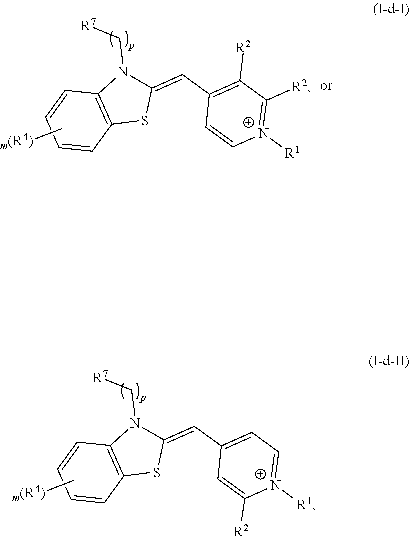

or a salt thereof. In some embodiments, the compound of Formula (I) is of Formula (I-d-I), or a salt thereof. In some embodiments, the compound of Formula (I) is of Formula (I-d-II), or a salt thereof.

In some embodiments, the compound of Formula (I) is of Formula (I-d), (I-d-I), or (I-d-II), or a salt thereof, wherein R¹ is methyl; and R² is selected from

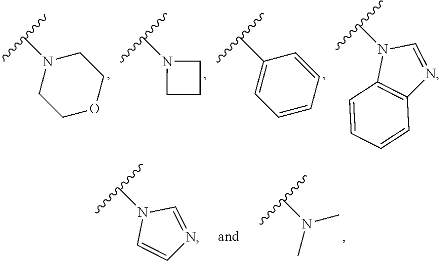

or two adjacent R² groups combine to form

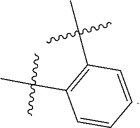 [SMILES: *c1ccccc1*]

m

As generally described herein, m is 0-4. In certain embodiments, m is 0, 1, 2, 3, or 4. In certain embodiments, m is 0, 1 or 2.

In some embodiments, m is 0. In some embodiments, m is 1. In some embodiments, m is 2. In some embodiments, m is 3. In some embodiments, m is 4.

In some embodiments, the compound of Formula (I) is of Formula (I-e):

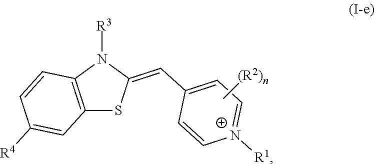

or a salt thereof.

In some embodiments, the compound of Formula (I) is of the Formula (I-e-I), (I-e-II), or (I-e-III):

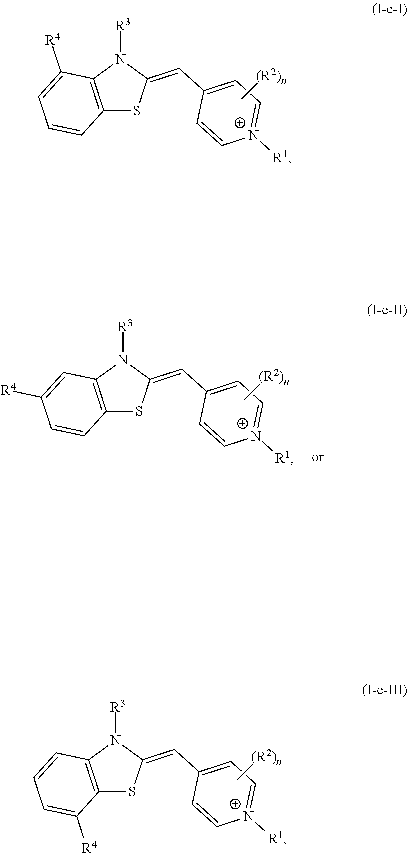

or a salt thereof.

In some embodiments, the compound of Formula (I) is of the Formula (I-e-IV), (I-e-V), (I-e-VI), (I-e-VII), (I-e-VIII), (I-e-IX), (I-e-X), or (I-e-XI):

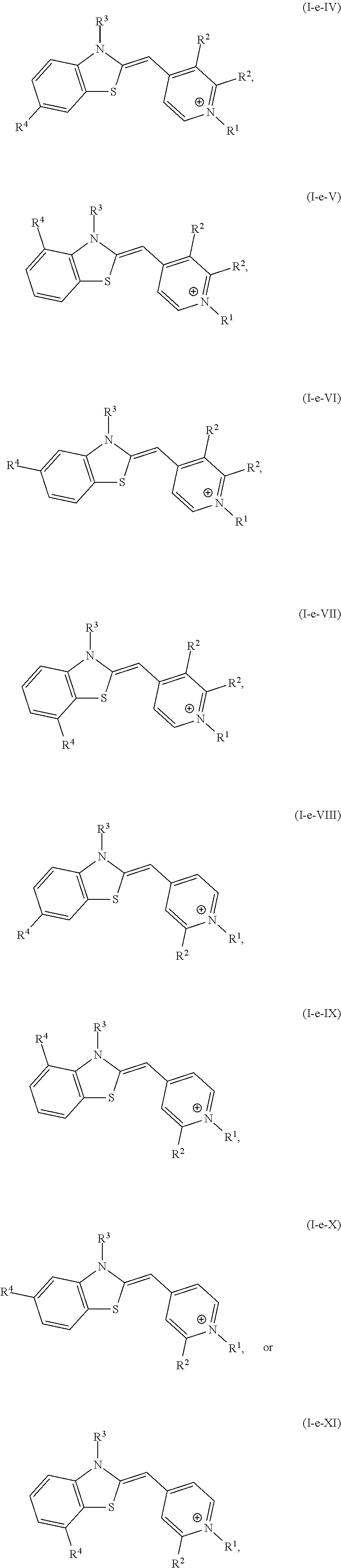

or a salt thereof.

In some embodiments, the compound of Formula (I) is of the Formula (I-e-I), or a salt thereof. In some embodiments, the compound of Formula (I) is of the Formula (I-e-II), or a salt thereof. In some embodiments, the compound of Formula (I) is of the Formula (I-e-III), or a salt thereof. In some embodiments, the compound of Formula (I) is of the Formula (I-e-IV), or a salt thereof. In some embodiments, the compound of Formula (I) is of the Formula (I-e-V), or a salt thereof. In some embodiments, the compound of Formula (I) is of the Formula (I-e-VI), or a salt thereof. In some embodiments, the compound of Formula (I) is of the Formula (I-e-VII), or a salt thereof. In some embodiments, the compound of Formula (I) is of the Formula (I-e-VIII), or a salt thereof. In some embodiments, the compound of Formula (I) is of the Formula (I-e-IX), or a salt thereof. In some embodiments, the compound of Formula (I) is of the Formula (I-e-X), or a salt thereof. In some embodiments, the compound of Formula (I) is of the Formula (I-e-XI), or a salt thereof.

In some embodiments, the compound of Formula (I) is of Formula (I-f):

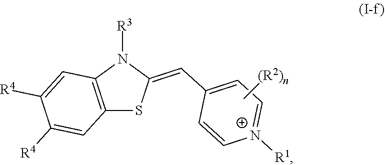

or a salt thereof. In some embodiments, the compound of Formula (I) is of Formula (I-f), or a salt thereof, wherein R¹ is hydrogen, substituted or unsubstituted alkyl, or substituted or unsubstituted heteroalkyl; R² is selected from

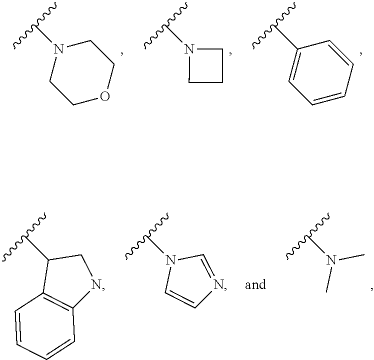

or two adjacent R² groups combine to form

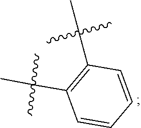 [SMILES: *c1ccccc1*]

and R³ is unsubstituted alkyl.

In some embodiments, the compound of Formula (I) is of Formula (I-f-I) or (I-f-II):

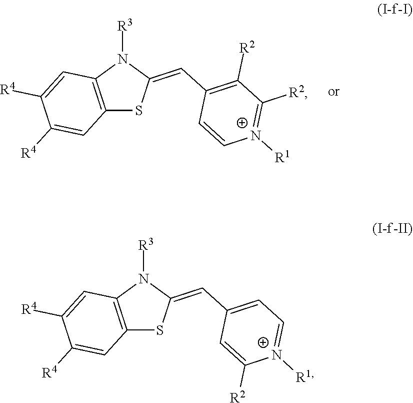

or a salt thereof. In some embodiments, the compound of Formula (I) is of Formula (I-f-I), or a salt thereof. In some embodiments, the compound of Formula (I) is of Formula (I-f-II), or a salt thereof.

In some embodiments, the compound of Formula (I) is of the Formula (I-e), (I-e-I), (I-e-II), (I-e-III), (I-e-IV), (I-e-V), (I-e-VI), (I-e-VII), (I-e-VIII), (I-e-IX), (I-e-X), (I-e-XI), (I-f), (I-f-I) or (I-f-II), or a salt thereof, wherein R¹ is hydrogen, substituted or unsubstituted alkyl, or substituted or unsubstituted heteroalkyl; R² is selected from

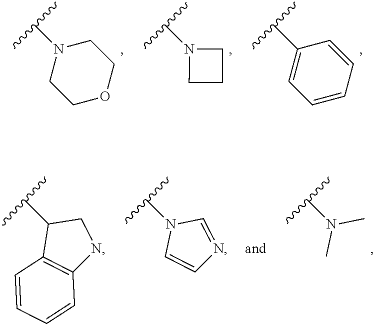

or two adjacent R² groups combine to form

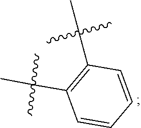 [SMILES: *c1ccccc1*]

and R³ is unsubstituted alkyl.

### R⁴

As generally described herein, R⁴ is, independently for each occurrence, halo, —NO₂, —OR⁶, —SR⁶, —N(R⁶)₂, substituted or unsubstituted alkyl, substituted or unsubstituted heteroalkyl, substituted or unsubstituted carbocyclyl, substituted or unsubstituted heterocyclyl, substituted or unsubstituted aryl, or substituted or unsubstituted heteroaryl. In certain embodiments, at least one instance of R⁴ is —NO₂. In certain embodiments, at least one instance of R⁴ is —SR⁶. In certain embodiments, at least one instance of R⁴ is —N(R⁶)₂. In certain embodiments, at least one instance of R⁴ is substituted or unsubstituted alkyl. In certain embodiments, at least one instance of R⁴ is substituted or unsubstituted heteroalkyl. In certain embodiments, at least one instance of R⁴ is substituted or unsubstituted carbocyclyl. In certain embodiments, at least one instance of R⁴ is substituted or unsubstituted heterocyclyl. In certain embodiments, at least one instance of R⁴ is substituted or unsubstituted aryl. In certain embodiments, at least one instance of R⁴ is substituted or unsubstituted heteroaryl.

In certain embodiments, at least one instance of R⁴ is substituted or unsubstituted alkyl, halo, or —OR⁶. In certain embodiments, at least one instance of R⁴ is substituted or unsubstituted C₁₋₆ alkyl. In certain embodiments, at least one instance of R⁴ is substituted C₁₋₆ alkyl. In certain embodiments, at least one instance of R⁴ is C₁₋₆ alkyl substituted with halo or quaternary ammonium. In certain embodiments, at least one instance of R⁴ is C₁₋₆ alkyl substituted with at least one fluoro. In certain embodiments, at least one instance of R⁴ is C₁₋₃ alkyl substituted with at least one fluoro. In certain embodiments, at least one instance of R⁴ is —CF₃. In certain embodiments, at least one instance of R⁴ is perhaloalkyl. In certain embodiments, at least one instance of R⁴ is unsubstituted C₁₋₆ alkyl.

In certain embodiments, at least one instance of R⁴ is —OR⁶.

In some embodiments, at least one R⁴ is halo. In some embodiments, at least one R⁴ is bromo. In some embodiments, at least one R⁴ is chloro. In some embodiments, at least one R⁴ is iodo. In some embodiments, at least one R⁴ is fluoro.

In some embodiments, the compound of Formula (I) is of the Formula (I-e), (I-e-I), (I-e-II), (I-e-III), (I-e-IV), (I-e-V), (I-e-VI), (I-e-VII), (I-e-VIII), (I-e-IX), (I-e-X), (I-e-XI), (I-f), (I-f-I) or (I-f-II), or a salt thereof, wherein R¹ is hydrogen, substituted or unsubstituted alkyl, or substituted or unsubstituted heteroalkyl; R² is selected from

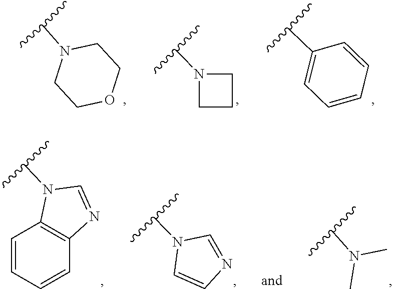

or two adjacent R² groups combine to form

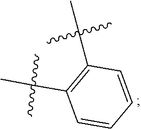 [SMILES: *c1ccccc1*]

R³ is unsubstituted alkyl; and at least one instance of R⁴ is substituted or unsubstituted alkyl, halo, or —OR⁶.

### R⁶

As generally described herein, R⁶ is, independently for each occurrence, hydrogen, substituted or unsubstituted alkyl, nitrogen protecting group when attached to a nitrogen, oxygen protecting group when attached to an oxygen, or a sulfur protecting group when attached to a sulfur. In some embodiments, each R⁶ is independently substituted or unsubstituted C₁₋₆ alkyl.

In certain embodiments, each R⁶ is independently C₁₋₆ alkyl substituted with a quaternary ammonium. In some embodiments, each R⁶ is independently C₁₋₆ alkyl substituted with a trimethylammonium. In certain embodiments, each R⁶ is independently methyl.

In some embodiments, the compound of Formula (I) is of the Formula (I-e), (I-e-I), (I-e-II), (I-e-III), (I-e-IV), (I-e-V), (I-e-VI), (I-e-VII), (I-e-VIII), (I-e-IX), (I-e-X), (I-e-XI), (I-f), (I-f-I) or (I-f-II), or a salt thereof, wherein R¹ is substituted or unsubstituted alkyl; at least one instance of R² is

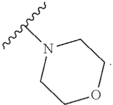 [SMILES: *N1CCOCC1]

R³ is unsubstituted alkyl; and at least one instance of R⁴ is —OMe.

In some embodiments, the compound of Formula (I) is selected from the group consisting of

| Compound Number | Structure |
|---|---|
| 1 | 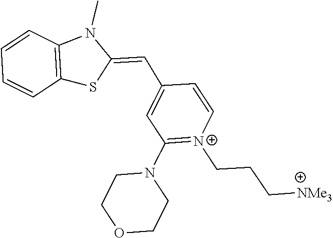 |
| 2 | 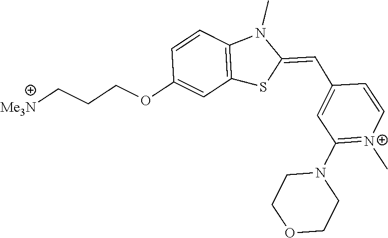 |
| 3 | 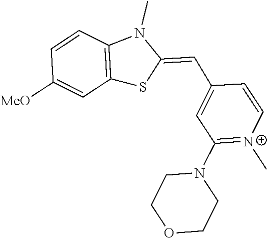 [SMILES: COc1ccc2c(c1)S/C(=C\c1cc[n+](C)c(N3CCOCC3)c1)N2C] |
| 4 | 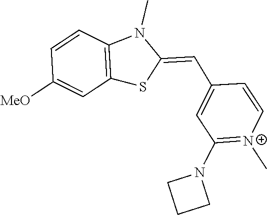 [SMILES: COc1ccc2c(c1)S/C(=C\c1cc[n+](C)c(N3CCC3)c1)N2C] |
| 5 | 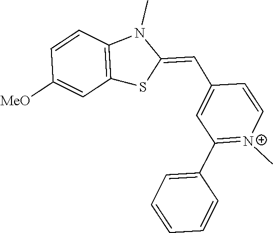 [SMILES: COc1ccc2c(c1)S/C(=C\c1cc[n+](C)c(-c3ccccc3)c1)N2C] |
| 6 | 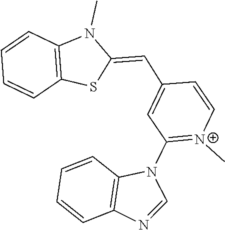 [SMILES: CN1/C(=C/c2cc[n+](C)c(-n3cnc4ccccc43)c2)Sc2ccccc21] |
| 7 | 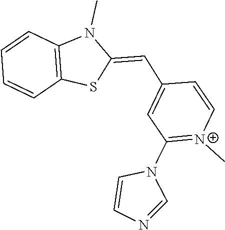 [SMILES: CN1/C(=C/c2cc[n+](C)c(-n3ccnc3)c2)Sc2ccccc21] |
| 8 | 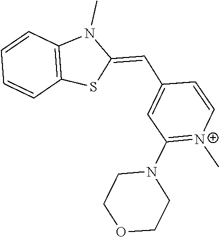 [SMILES: CN1/C(=C/c2cc[n+](C)c(N3CCOCC3)c2)Sc2ccccc21] |
| 9 | 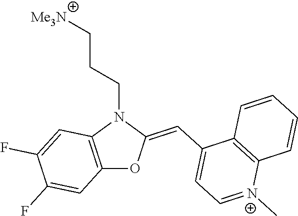 |
| 10 | 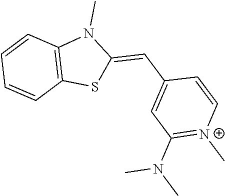 [SMILES: CN(C)c1cc(/C=C2\Sc3ccccc3N2C)cc[n+]1C] |
| 11 | 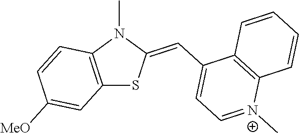 [SMILES: COc1ccc2c(c1)S/C(=C\c1cc[n+](C)c3ccccc13)N2C] |

or a salt thereof. In certain embodiments, the preceding compounds of Formula (I) further comprise an acetate anion.

In some embodiments, the compound of Formula (I), or salt thereof, fluoresces at about 488 nm excitation wavelength. In some embodiments, the compound of Formula (I), or salt thereof, fluoresces at 488 nm excitation wavelength. In some embodiments, the compound of Formula (I), or salt thereof, fluoresces at about 498 nm excitation wavelength. In some embodiments, the compound of Formula (I), or salt thereof, fluoresces at 498 nm excitation wavelength. In some embodiments, the compound of Formula (I), or salt thereof, fluoresces at about 440-500 nm excitation wavelength. In some embodiments, the compound of Formula (I), or salt thereof, fluoresces at about 440-460 nm excitation wavelength. In some embodiments, the compound of Formula (I), or salt thereof, fluoresces at about 460-480 nm excitation wavelength. In some embodiments, the compound of Formula (I), or salt thereof, fluoresces at about 480-500 nm excitation wavelength. In some embodiments, the compound of Formula (I), or salt thereof, has a λ_{abs} of 448 and λₑₘ of 498 nm. In some embodiments, the compound of Formula (I), or salt thereof, has a λ_{abs} of 448. In some embodiments, the compound has a λₑₘ of 498 nm.

In certain embodiments, the compound of Formula (I), or salt thereof, does not fluoresce at about 520 nm or about 638 nm excitation wavelengths.

In some embodiments, the compound of Formula (I), or salt thereof, is stable in aqueous solution. In some embodiments, the compound of Formula (I), or salt thereof, is 100% water soluble. In some embodiments, the compound of Formula (I), or salt thereof, is water soluble. In some embodiments, the compound of Formula (I), or salt thereof, is at least 75% water soluble. In some embodiments, the compound of Formula (I), or salt thereof, is at least 85% water soluble. In some embodiments, the compound of Formula (I), or salt thereof, is at least 90% water soluble. In some embodiments, more than about 95% of compound of Formula (I), or salt thereof, remains after stored for ≥30 days. In some embodiments, more than about 80% of compound of Formula (I), or salt thereof, remains after stored for ≥30 days. In some embodiments, more than about 95% of compound of Formula (I), or salt thereof, remains after stored for ≥60 days. In some embodiments, more than about 80% of compound of Formula (I), or salt thereof, remains after stored for ≥60 days.

In certain embodiments, the compound of Formula (I), or salt thereof, is stable in lyophilized form. In certain embodiments, the compound of Formula (I), or salt thereof, is stable in lyophilized form after storage for ≥30 days. In certain embodiments, the compound of Formula (I), or salt thereof, is stable in lyophilized form after storage for ≥60 days.

In some embodiments, the compound of Formula (I), or salt thereof, is stable at about 15-30° C.

In certain embodiments, the compound of Formula (I), or salt thereof, is stable at about 15-30° C. for at least one month.

In some embodiments, the compound of Formula (I), or salt thereof, is stable at about 15-30° C. for 6 months. In some embodiments, the compound of Formula (I), or salt thereof, is stable at about 15-30° C., in lyophilized form for ≥6 months. In some embodiments, the compound of Formula (I), or salt thereof, is stable at about 15-30° C., in lyophilized form for ≥1 month.

In some embodiments, the compound of Formula (I), or salt thereof, is stable at about 30-50° C.

In certain embodiments, the compound of Formula (I), or salt thereof, is stable at about 30-50° C. for at least one month.

In some embodiments, the compound of Formula (I), or salt thereof, is stable at about 30-50° C. for 6 months.

In some embodiments, the compound of Formula (I), or salt thereof, is compatible with LAMP conditions. In some embodiments, the compound of Formula (I), or salt thereof, does not inhibit LAMP primers.

In another aspect of the present disclosure, provided is a composition comprising a compound of Formula (I) described herein, or a salt thereof.

In some embodiments, the composition does not comprise a compound comprising a quaternary ammonium group.

In certain embodiments, the composition is a solution or a suspension. In certain embodiments, the composition is a solution. In certain embodiments, the composition is a suspension. In some embodiments, the concentration of the compound of Formula (I), or salt thereof, in the composition is in the range of about 0.001-100 μM. In certain embodiments, the concentration of the compound of Formula (I), or salt thereof, in the composition is in the range of about 0.001-100 μM. In certain embodiments, the concentration of the compound of Formula (I), or salt thereof, in the composition is in the range of about 0.001-0.01 μM. In certain embodiments, the concentration of the compound of Formula (I), or salt thereof, in the composition is in the range of about 0.01-0.1 μM. In certain embodiments, the concentration of the compound of Formula (I), or salt thereof, in the composition is in the range of about 0.1-1 μM. In certain embodiments, the concentration of the compound of Formula (I), or salt thereof, in the composition is in the range of about 1-10 μM. In certain embodiments, the concentration of the compound of Formula (I), or salt thereof, in the composition is in the range of about 10-100 μM.

In certain embodiments, the composition further comprises a test sample. In some embodiments, the test sample is of an animal bodily tissue or fluid. In some embodiments, the test sample is of an animal bodily tissue. In some embodiments, the test sample is of an animal bodily fluid. In certain embodiments, the animal bodily tissue or fluid is of a human.

In some embodiments, the test sample comprises an analyte.

In certain embodiments, the analyte is a nucleic acid. In some embodiments, the nucleic acid is RNA. In certain embodiments, the nucleic acid is DNA. In some embodiments, the DNA is double-stranded DNA. In certain embodiments, the DNA is single-stranded DNA.

In certain embodiments, the nucleic acid is of a virus or bacterium. In certain embodiments, the virus or bacterium is a sexually transmitted infection.

In some embodiments, the virus is SARS-CoV-2. In some embodiments, the virus is influenza. In some embodiments, the influenza is influenza B. In some embodiments, the influenza is H1N1.

In certain embodiments, the bacterium is Neisseria gonorrhoeae or Chlamydia trachomatis.

In some embodiments, the composition has a Stokes shift of between about 30 and about 60 nm. In some embodiments, the composition has a Stokes shift of greater than about 30 nm. In some embodiments, the composition has a Stokes shift of greater than about 35 nm. In some embodiments, the composition has a Stokes shift of greater than about 40 nm. In some embodiments, the composition has a Stokes shift of greater than about 45 nm. In some embodiments, the composition has a Stokes shift of greater than about 50 nm. In some embodiments, the composition has a Stokes shift of greater than about 55 nm. In certain embodiments, the composition has a Stokes shift of about 50 nm. In some embodiments, the composition has a Stokes shift of 50 nm.

### Methods

In one aspect, provided is a method of detecting the presence or absence of an analyte in a test sample, comprising contacting the test sample with a compound of Formula (I), or a salt thereof, in the presence of an energy (E1) and detecting emission of an energy (E2) from the sample.

In some embodiments, the compound of Formula (I), or salt thereof, does not contain a quaternary ammonium group.

In certain embodiments, the method comprises a compound of Formula (I), or salt thereof, containing a quaternary ammonium group, at a concentration of less than about 2 μM.

In some embodiments, the test sample is of an animal bodily tissue or fluid. In some embodiments, the test sample is of an animal bodily tissue. In some embodiments, the test sample is of an animal bodily fluid.

In certain embodiments, the animal bodily tissue or fluid is of a human.

In some embodiments, the test sample is blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, or swabs (such as buccal swabs).

In some embodiments, the test sample comprises an analyte. In certain embodiments, the analyte is a nucleic acid. In some embodiments, the nucleic acid is RNA. In some embodiments, the nucleic acid is DNA. In certain embodiments, the DNA is double-stranded DNA. In certain embodiments, the DNA is single-stranded DNA.

In some embodiments, the nucleic acid is of a virus or bacterium. In certain embodiments, the virus is SARS-CoV-2. In some embodiments, the bacterium is Neisseria gonorrhoeae or Chlamydia trachomatis.

In certain embodiments, E1 is electromagnetic radiation having a wavelength of about 400 nm to about 800 nm. In certain embodiments, E1 is electromagnetic radiation having a wavelength of about 400 nm to about 500 nm. In certain embodiments, E1 is electromagnetic radiation having a wavelength of about 440 nm to about 500 nm. In certain embodiments, E1 is electromagnetic radiation having a wavelength of about 440 nm to about 460 nm. In certain embodiments, E1 is electromagnetic radiation having a wavelength of about 460 nm to about 480 nm. In certain embodiments, E1 is electromagnetic radiation having a wavelength of about 480 nm to about 500 nm. In certain embodiments, E1 is electromagnetic radiation having a wavelength of about 488 nm. In certain embodiments, E1 is electromagnetic radiation having a wavelength of about 498 nm. In some embodiments, E1 is electromagnetic radiation having a wavelength of about 442 nm, about 443 nm, about 448 nm, about 469 nm, or about 510 nm.

In some embodiments, E2 is emitted by fluorescence of the compound. In some embodiments, E2 is electromagnetic radiation having a wavelength of about 460 nm to about 600 nm. In some embodiments, E2 is electromagnetic radiation having a wavelength of about 480 nm to about 600 nm. In some embodiments, E2 is electromagnetic radiation having a wavelength of about 460 nm to about 530 nm. In some embodiments, E2 is electromagnetic radiation having a wavelength of about 520 nm to about 600 nm. In some embodiments, E2 is electromagnetic radiation having a wavelength of about 480 nm, about 481 nm, about 493 nm, about 498 nm, about 499 nm, or about 553 nm. In certain embodiments, E2 of the test sample is lower when the analyte is present. In some embodiments, E2 of the test sample is higher when the analyte is present.

In certain embodiments, the test sample is a solution or suspension. In some embodiments, the compound of Formula (I), or salt thereof, is present in an amount of about 1-10 nmol per milliliter of test sample. In some embodiments, the compound is present in an amount of about 1-5 nmol per milliliter of test sample. In some embodiments, the compound is present in an amount of about 5-10 nmol per milliliter of test sample.

In certain embodiments, the temperature is greater than about 61° C. In certain embodiments, the temperature is about 60° C.

In some embodiments, the method is LAMP, gel staining, PCR, or qPCR. In some embodiments, the method is LAMP. In some embodiments, the method is a nucleic acid detection technique. In some embodiments, the method is a fluorescence detection technique. In some embodiments, the method is a nucleic acid quantification assay. In some embodiments, the method comprises nucleic acid staining.

In another aspect, the present disclosure provides a kit comprising: a compound disclosed herein; or a composition of the present disclosure; and instructions for use.

### EXAMPLES

### Example 1. Synthetic Procedures

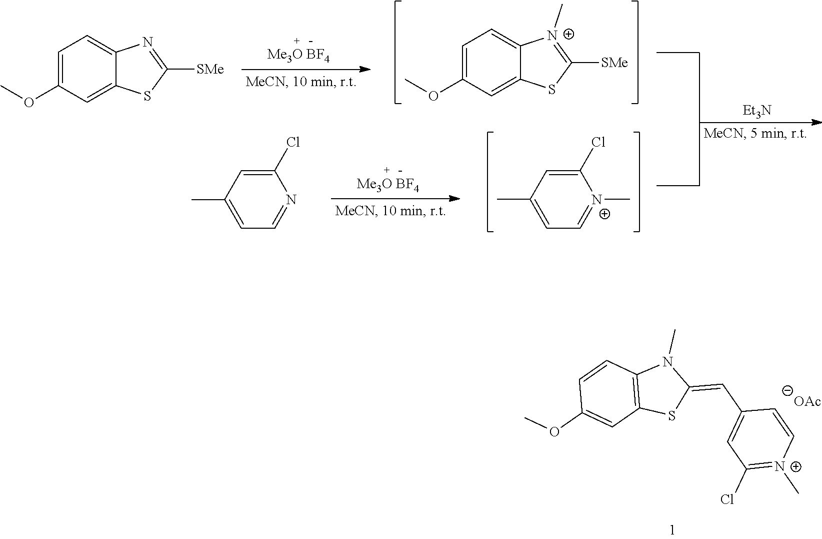

Synthesis of Intermediate 1. To a scintillation vial was added 6-methoxy-2-(methylthio)benzo[d]thiazole (107 mg, 0.506 mmol), MeCN (2.00 mL), and trimethyloxonium tetrafluoroborate (79 mg, 0.534 mmol). The reaction was stirred at room temperature (r.t.) for 10 min. Separately, a scintillation vial containing 2-chloro-4-methylpyridine (70 μL, 0.627 mmol) in MeCN (2.00 mL) was added trimethyloxonium tetrafluoroborate (97 mg, 0.656 mmol), and the mixture was stirred for 10 min. The two reactions were mixed and triethylamine (0.21 mL, 1.51 mmol) was added and stirred for 5 min. The reaction mixture was quenched with H₂O and directly purified by reversed phase chromatography (0 to 100% MeOH/H₂O 0.1% TEAA, Hamilton PRP C₁₈ 21.1×250 mm column). Evaporation of the product containing fractions afforded 1 (74 mg, 39% yield) as an orange solid. HRMS (ESI) calculated for C₁₆H₁₆ClN₂OS⁺ (M)⁺319.0666, observed 319.0678.

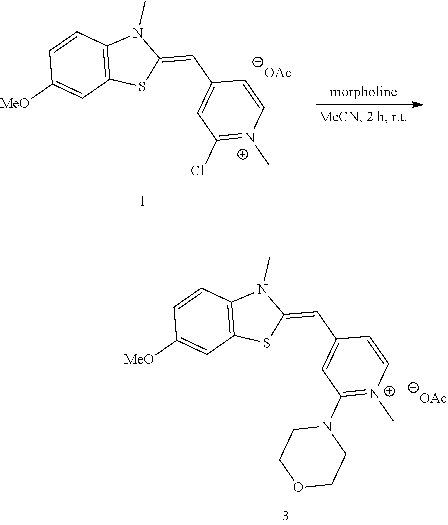 [SMILES: COc1ccc2c(c1)S/C(=C\c1cc[n+](C)c(Cl)c1)N2C ; COc1ccc2c(c1)S/C(=C\c1cc[n+](C)c(N3CCOCC3)c1)N2C ; "OAc" ; "OAc"]

Synthesis of Compound 3. To a solution of 1 (42 mg, 0.111 mmol) in MeCN (2.00 mL) was added morpholine (0.19 mL, 2.17 mmol) at room temperature and stirred for 2 h. The reaction mixture was diluted with H₂O and directly purified by reversed phase chromatography (0 to 100% MeOH/H₂O 0.1% TEAA, Hamilton PRP C₁₈ 21.1×250 mm column). Evaporation of the product containing fractions afforded compound 3 (as the acetate salt) (19 mg, 40% yield) as an orange solid. HRMS (ESI) calculated for C₂₀H₂₄N₃O₂S⁺ (M)⁺370.1584, observed 370.1586.

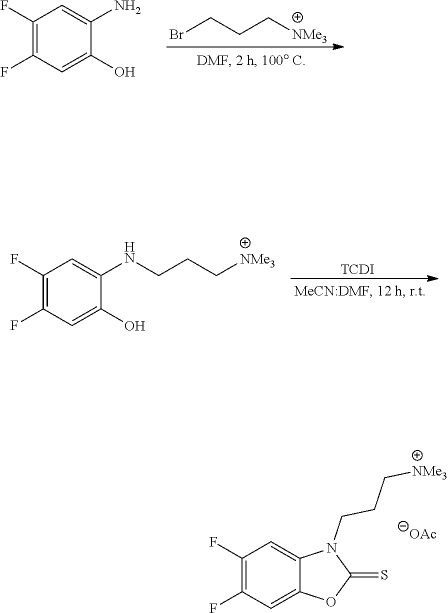

Synthesis of Intermediate to Compound 9. To a scintillation vial was added 2-amino-4,5-difluorophenol (200 mg, 1.38 mmol), DMF (2.00 mL), and (3-bromopropyl)trimethylammonium bromide (363 mg, 1.39 mmol). The reaction was stirred at 100° C. for 2 h, cooled to r.t., and solution of 1,1-thiocarbonyldiimidazole (TCDI) (246 mg, 1.38) in MeCN (3 mL) was added dropwise and mixture was stirred at r.t. for 12 h. The reaction mixture was quenched with H₂O and directly purified by reversed phase chromatography (0 to 100% MeOH/H₂O 0.1% TEAA, Hamilton PRP C₁₈ 21.1×250 mm column). Evaporation of the product containing fractions afforded the desired compound and used directly in the next step.

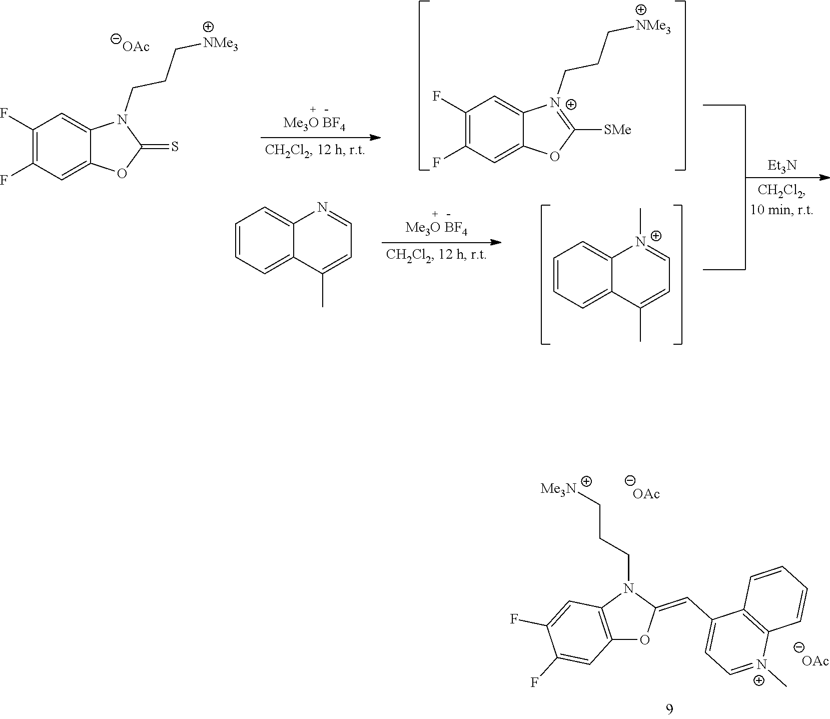

Synthesis of Compound 9. To a scintillation vial was added 3-(5,6-difluoro-2-thioxobenzo[d]oxazol-3(2H)-yl)-N,N,N-trimethylpropan-1-aminium acetate (103 mg, 0.297 mmol), CH₂Cl₂ (1.00 mL), and trimethyloxonium tetrafluoroborate (53 mg, 0.358 mmol). The reaction was stirred at r.t. for 12 hr. Separately, a scintillation vial containing 4-methylquinoline (47 μL, 0.358 mmol) in CH₂Cl₂ (1.00 mL) was added trimethyloxonium tetrafluoroborate (53 mg, 0.358 mmol), and the mixture was stirred for 12 hr. The two reactions were mixed, and triethylamine (0.15 mL, 1.08 mmol) was added and stirred for 10 min. The reaction mixture was quenched with H₂O and directly purified by reversed phase chromatography (0 to 100% MeOH/H₂O 0.1% TEAA, Hamilton PRP C₁₈ 21.1×250 mm column). Evaporation of the product containing fractions afforded compound 9 (as the diacetate salt) as a dark orange solid. HRMS (ESI) calculated for C₂₄H₂₇F₂N₃O²⁺ (M)²⁺205.6056, observed 205.6064.

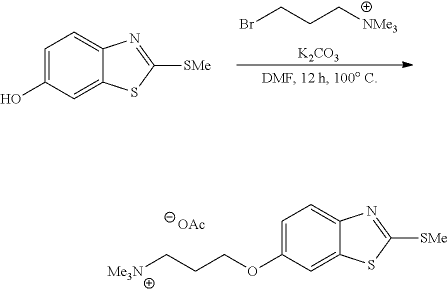

Synthesis of Intermediate to Compound 2. To a scintillation vial was added 2-(methylthio)benzo[d]thiazol-6-ol (200 mg, 1.01 mmol), DMF (2.00 mL), (3-bromopropyl)trimethylammonium bromide (316 mg, 1.21 mmol), and potassium carbonate (350 mg, 2.53 mmol). The reaction was stirred at 100° C. for 12 h. The reaction mixture was quenched with H₂O and directly purified by reversed phase chromatography (0 to 100% MeOH/H₂O 0.1% TEAA, Hamilton PRP C₁₈ 21.1×250 mm column). Evaporation of the product containing fractions afforded the desired compound and used directly in the next step.

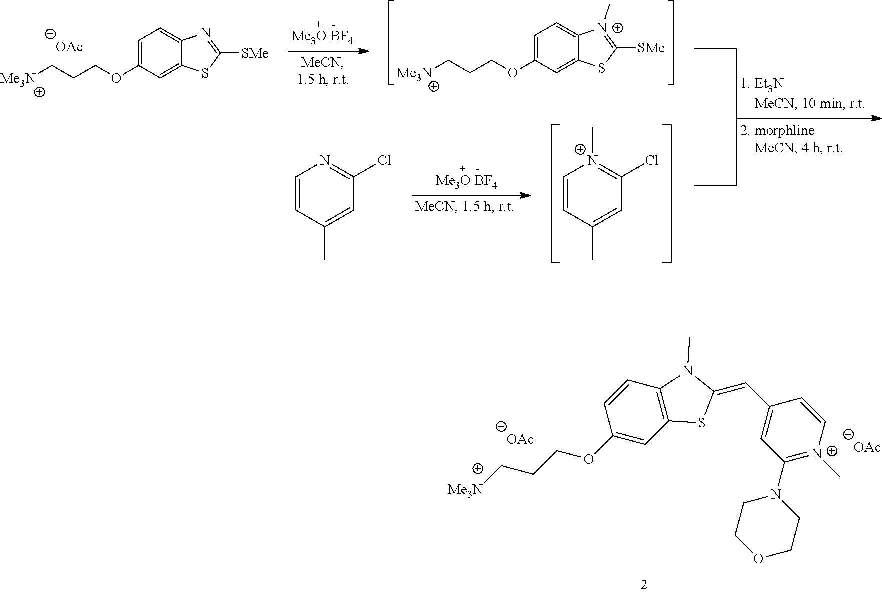

Synthesis of Compound 2. To a scintillation vial was added N,N,N-trimethyl-3-((2-(methylthio)benzo[d]thiazol-6-yl)oxy)propan-1-aminium acetate (46 mg, 0.129 mmol), MeCN (1.00 mL), and trimethyloxonium tetrafluoroborate (20 mg, 0.135 mmol). The reaction was stirred at r.t. for 1.5 hr. Separately, a scintillation vial containing 2-chloro-4-methylpyridine (45 μL, 0.403 mmol) in MeCN (1.00 mL) was added trimethyloxonium tetrafluoroborate (60 mg, 0.405 mmol), and the mixture was stirred for 1.5 hr. The two reactions were mixed, and triethylamine (54 μL, 0.387 mmol) was added and stirred for 10 min. The reaction mixture was quenched with H₂O and directly purified by reversed phase chromatography (0 to 100% MeOH/H₂O 0.1% TEAA, Hamilton PRP C₁₈ 21.1×250 mm column). Evaporation of the product containing fractions afforded desired intermediate. To a solution of intermediate in MeCN (1 mL) was added morpholine (0.12 mL, 1.37 mmol) at room temperature and stirred for 4 h. The reaction mixture was diluted with H₂O and directly purified by reversed phase chromatography (0 to 100% MeOH/H₂O 0.1% TEAA, Hamilton PRP C₁₈ 21.1×250 mm column). Evaporation of the product containing fractions afforded compound 2 (as the diacetate salt) as an orange solid. HRMS (ESI) calculated for C₂₅H₃₆N₄O₂S²⁺ (M)²⁺ 228.1274, observed 228.1286.

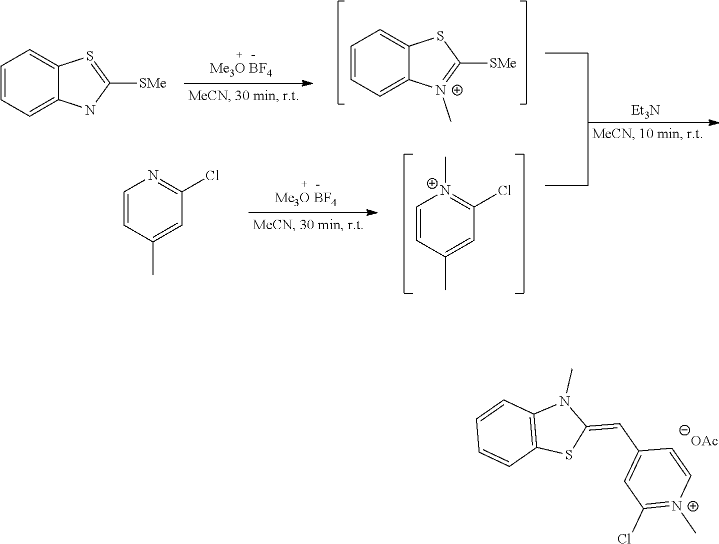

Synthesis of Intermediate to Compounds 7 and 8. To a scintillation vial was added 2-(methylthio)benzo[d]thiazole (142 mg, 0.783 mmol), MeCN (3.00 mL), and trimethyloxonium tetrafluoroborate (127 mg, 0.859 mmol). The reaction was stirred at r.t. for 30 min. Separately, a scintillation vial containing 2-chloro-4-methylpyridine (0.13 mL, 1.16 mmol) in MeCN (2.00 mL) was added trimethyloxonium tetrafluoroborate (180 mg, 1.22 mmol), and the mixture was stirred for 30 min. The two reactions were mixed, and triethylamine (0.33 mL, 2.37 mmol) was added and stirred for 10 min. The reaction mixture was quenched with H₂O and directly purified by reversed phase chromatography (0 to 100% MeOH/H₂O 0.1% TEAA, Hamilton PRP C₁₈ 21.1×250 mm column). Evaporation of the product containing fractions afforded the desired compound as an orange solid. HRMS (ESI) calculated for C₁₅H₁₄ClN₂S⁺ (M)⁺289.0561, observed 289.0572.

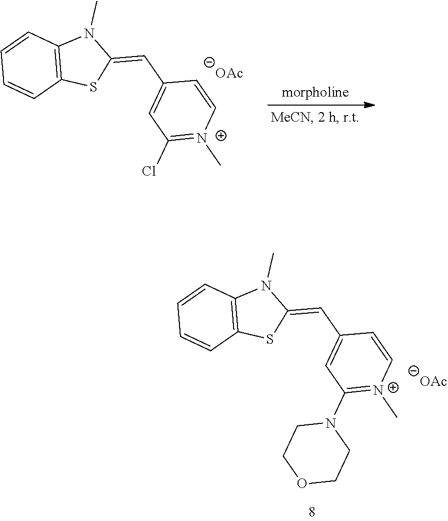 [SMILES: CN1/C(=C/c2cc[n+](C)c(Cl)c2)Sc2ccccc21 ; CN1/C(=C/c2cc[n+](C)c(N3CCOCC3)c2)Sc2ccccc21 ; "OAc" ; "OAc"]

Synthesis of Compound 8. To a solution of 2-chloro-1-methyl-4-((3-methylbenzo[d]thiazol-2(3H)-ylidene)methyl)pyridin-1-ium (78 mg, 0.224 mmol) in MeCN (2.00 mL) was added morpholine (0.4 mL, 4.57 mmol) at r.t. and stirred for 2 h. The reaction mixture was diluted with H₂O and directly purified by reversed phase chromatography (0 to 100% MeOH/H₂O 0.1% TEAA, Hamilton PRP C₁₈ 21.1×250 mm column). Evaporation of the product containing fractions afforded compound 8 (as the acetate salt) as an orange solid. HRMS (ESI) calculated for C₁₉H₂₂N₃OS⁺ (M)⁺340.1478, observed 340.1494.

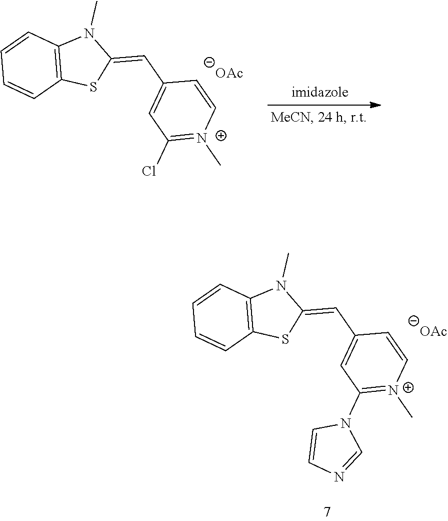 [SMILES: CN1/C(=C/c2cc[n+](C)c(Cl)c2)Sc2ccccc21 ; CN1/C(=C/c2cc[n+](C)c(-n3ccnc3)c2)Sc2ccccc21 ; "OAc" ; "OAc"]

Synthesis of Compound 7. To a solution of 2-chloro-1-methyl-4-((3-methylbenzo[d]thiazol-2(3H)-ylidene)methyl)pyridin-1-ium (60 mg, 0.172 mmol) in MeCN (3.00 mL) was added imidazole (234 mg, 3.43 mmol) at r.t. and stirred for 24 h. The reaction mixture was diluted with H₂O and directly purified by reversed phase chromatography (0 to 100% MeOH/H₂O 0.1% TEAA, Hamilton PRP C₁₈ 21.1×250 mm column). Evaporation of the product containing fractions afforded compound 7 (as the acetate salt) as an orange solid. HRMS (ESI) calculated for C₁₈H₁₇N₄S⁺ (M)⁺321.1168, observed 321.1169.

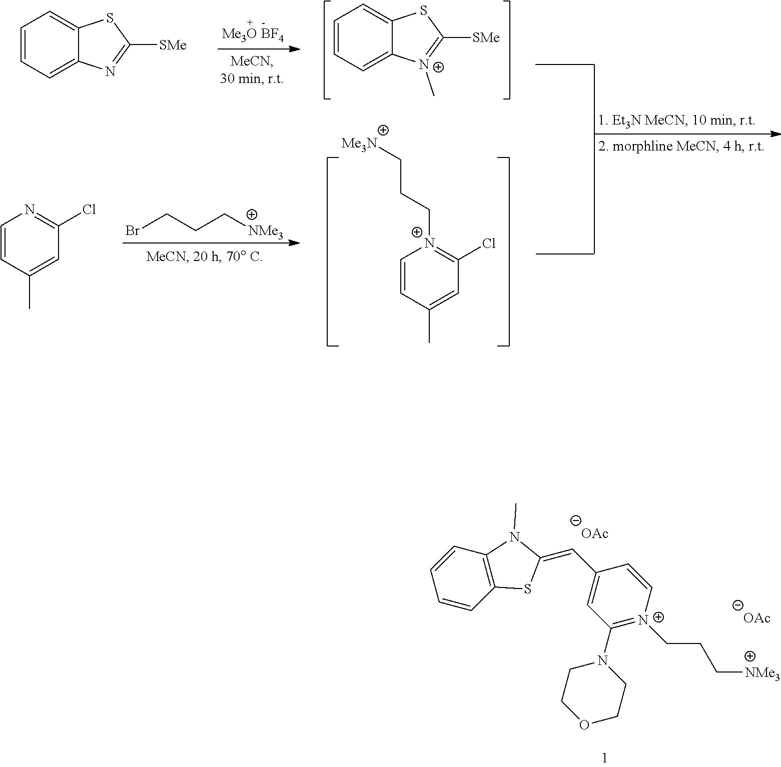

Synthesis of Compound 1. A scintillation vial containing 2-chloro-4-methylpyridine (0.17 mL, 1.52 mmol) in MeCN (2.00 mL) was added (3-bromopropyl)trimethylammonium bromide (200 mg, 0.766 mmol), and the mixture was stirred for 20 h at 70° C. Separately, a scintillation vial was added 2-(methylthio)benzo[d]thiazole (139 mg, 0.767 mmol), MeCN (2.00 mL), and trimethyloxonium tetrafluoroborate (115 mg, 0.777 mmol). The reaction was stirred at r.t. for 30 min. The two reactions were mixed, and triethylamine (0.32 mL, 2.30 mmol) was added and stirred for 10 min. The reaction mixture was quenched with H₂O and directly purified by reversed phase chromatography (0 to 100% MeOH/H₂O 0.1% TEAA, Hamilton PRP C₁₈ 21.1×250 mm column). Evaporation of the product containing fractions afforded desired intermediate. To a solution of intermediate in MeCN (1 mL) was added morpholine (0.2 mL, 2.29 mmol) at room temperature and stirred for 4 h. The reaction mixture was diluted with H₂O and directly purified by reversed phase chromatography (0 to 100% MeOH/H₂O 0.1% TEAA, Hamilton PRP C₁₈ 21.1×250 mm column). Evaporation of the product containing fractions afforded compound 1 (as the diacetate salt) as an orange solid. HRMS (ESI) calculated for C₂₄H₃₄N₄OS²⁺ (M)²⁺213.1221, observed 213.1232.

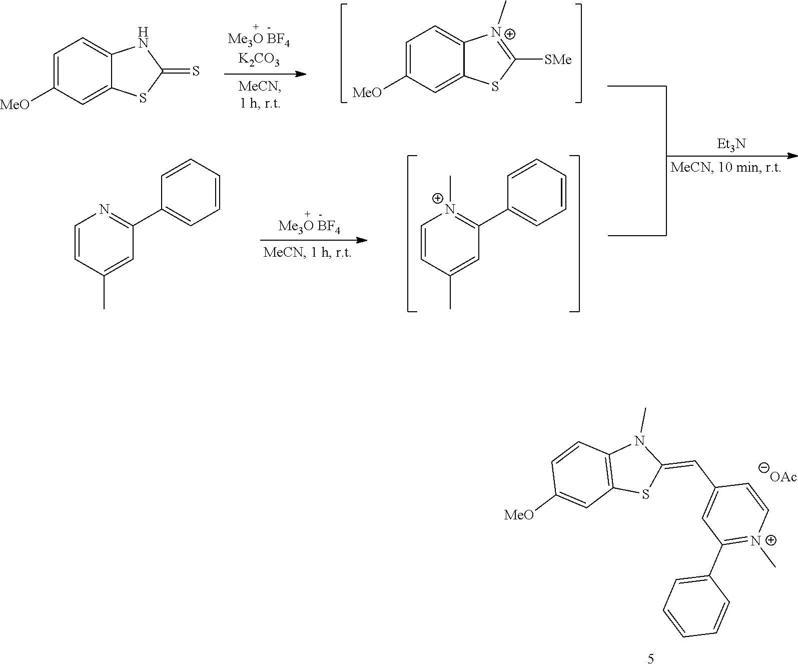

Synthesis of Compound 5. A scintillation vial containing 6-methoxybenzo[d]thiazole-2(3H)-thione (40 mg, 0.203 mmol) in MeCN (2.00 mL) was added trimethyloxonium tetrafluoroborate (63 mg, 0.426 mmol), potassium carbonate (28 mg, 0.203) and the mixture was stirred for 1 h. Separately, a scintillation vial was added 4-methyl-2-phenylpyridine (52 mg, 0.307 mmol), MeCN (2.00 mL), and trimethyloxonium tetrafluoroborate (50 mg, 0.338 mmol). The reaction was stirred at r.t. for 1 h. The two reactions were mixed, and triethylamine (0.1 mL, 0.717 mmol) was added and stirred for 10 min. The reaction mixture was quenched with H₂O and directly purified by reversed phase chromatography (0 to 100% MeOH/H₂O 0.1% TEAA, Hamilton PRP C₁₈ 21.1×250 mm column). Evaporation of the product containing fractions afforded compound 5 (as the acetate salt) as an orange solid. HRMS (ESI) calculated for C₂₂H₂₁N₂OS⁺ (M)⁺361.1369, observed 361.1387.

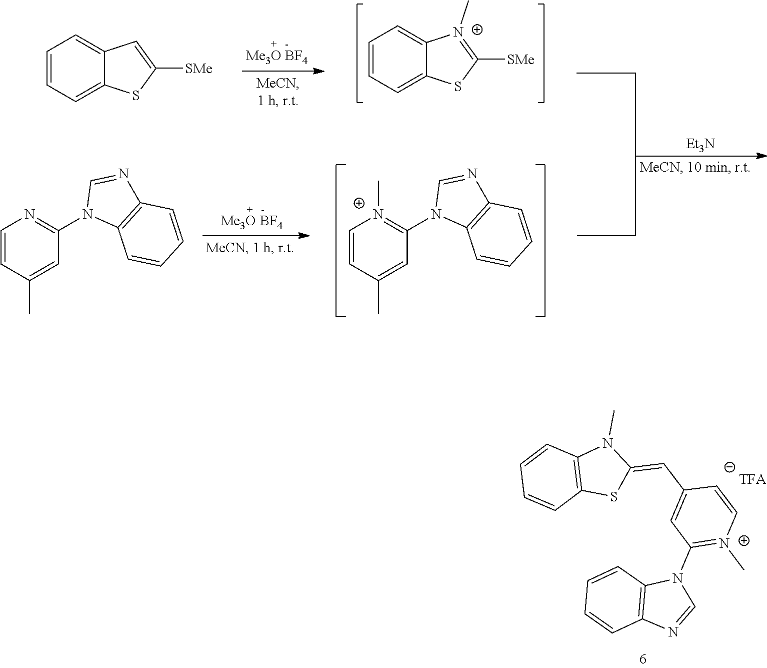

Synthesis of Compound 6. To a scintillation vial was added 2-(methylthio)benzo[d]thiazole (77 mg, 0.368 mmol), MeCN (3.00 mL), and trimethyloxonium tetrafluoroborate (120 mg, 0.811 mmol). The reaction was stirred at r.t. for 1 h. Separately, a scintillation vial containing 1-(4-methylpyridin-2-yl)-1H-benzo[d]imidazole (67 mg, 0.370 mmol) in MeCN (2.00 mL) was added trimethyloxonium tetrafluoroborate (60 mg, 0.406 mmol), and the mixture was stirred for 1 h. The two reactions were mixed, and triethylamine (0.16 mL, 1.15 mmol) was added and stirred for 10 min. The reaction mixture was quenched with H₂O and directly purified by reversed phase chromatography (0 to 100% MeOH/H₂O 0.1% TEAA, Hamilton PRP C₁₈ 21.1×250 mm column). A second purification was performed using 100% MeOH/H₂O 0.1% TFA. Evaporation of the product containing fractions afforded compound 6 (as the trifluoroacetate salt) as an orange solid. HRMS (ESI) calculated for C₂₂H₁₉N₄S⁺ (M)⁺371.1325, observed 371.1328.

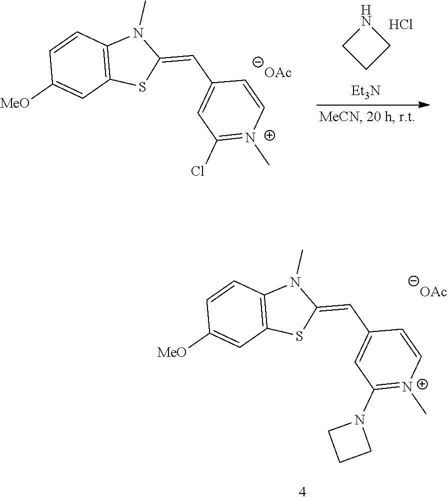

Synthesis of Compound 4. To a solution of 2-chloro-4-((6-methoxy-3-methylbenzo[d]thiazol-2(3H)-ylidene)methyl)-1-methylpyridin-1-ium (34 mg, 0.0897 mmol) in MeCN (2.00 mL) was added Azetidine hydrochloride (84 mg, 0.898 mmol) and DMSO (1 mL) and mixture was stirred at r.t. for 20 h. The reaction mixture was diluted with H₂O and directly purified by reversed phase chromatography (0 to 100% MeOH/H₂O 0.1% TEAA, Hamilton PRP C₁₈ 21.1×250 mm column). Evaporation of the product containing fractions afforded compound 4 (as the acetate salt) as an orange solid. HRMS (ESI) calculated for C₁₉H₂₂N₃OS⁺ (M)⁺340.1478, observed 340.1498.

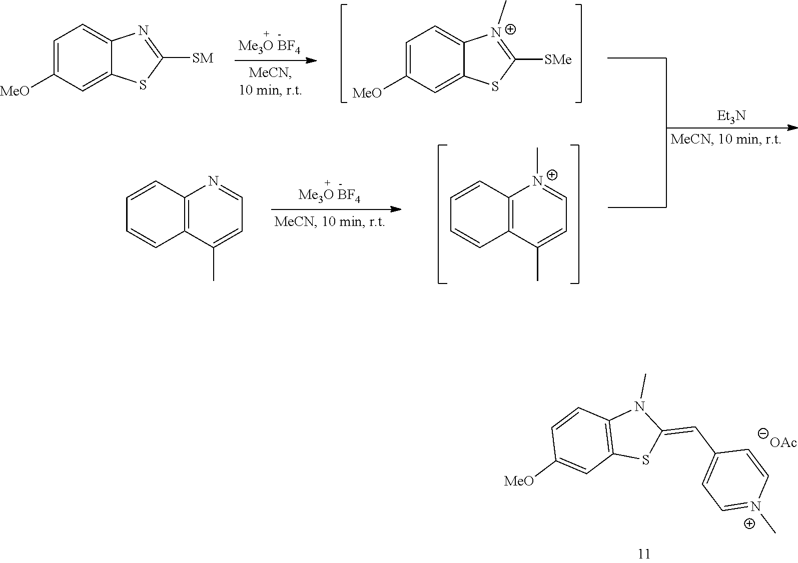

Synthesis of Compound 11. To a scintillation vial was added 6-methoxy-2-(methylthio)benzo[d]thiazole (66 mg, 0.312 mmol), MeCN (2.00 mL), and trimethyloxonium tetrafluoroborate (48 mg, 0.325 mmol). The reaction was stirred at r.t. for 10 min. Separately, a scintillation vial containing 4-methylquinoline (51 μL, 0.386 mmol) in MeCN (2.00 mL) was added trimethyloxonium tetrafluoroborate (60 mg, 0.406 mmol), and the mixture was stirred for 10 min. The two reactions were mixed, and triethylamine (0.13 mL, 0.933 mmol) was added and stirred for 10 min. The reaction mixture was quenched with H₂O and directly purified by reversed phase chromatography (0 to 100% MeOH/H₂O 0.1% TEAA, Hamilton PRP C₁₈ 21.1×250 mm column). Evaporation of the product containing fractions afforded compound 11 (as the acetate salt) as an orange solid. HRMS (ESI) calculated for C₂₀H₁₉N₂OS⁺ (M)⁺335.1213, observed 335.1212.

### Example 2. LAMP Assays and Characterization

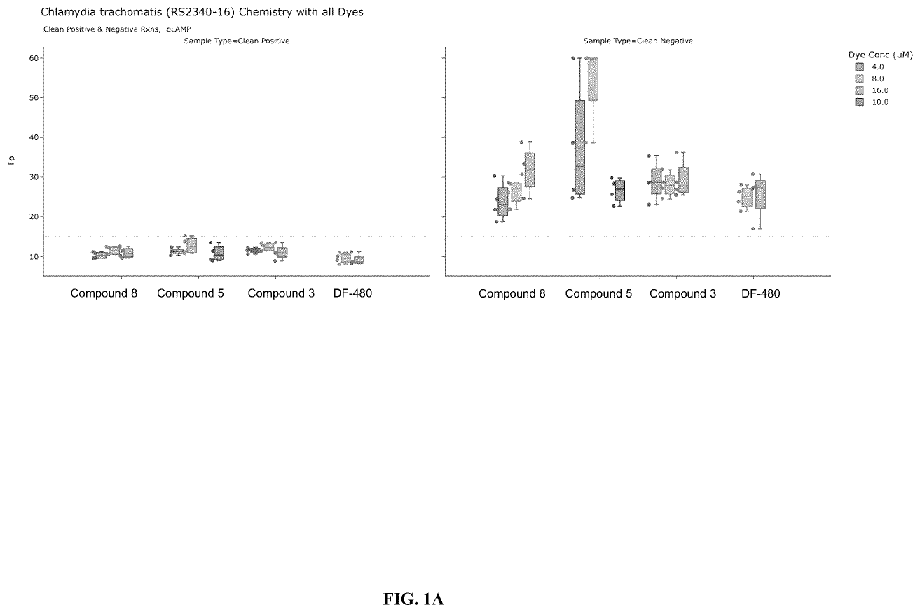

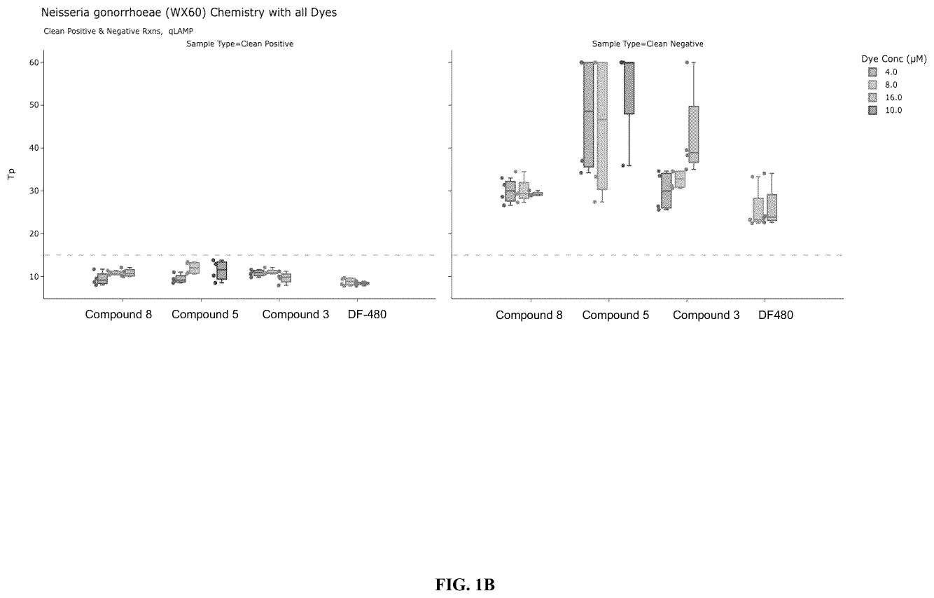

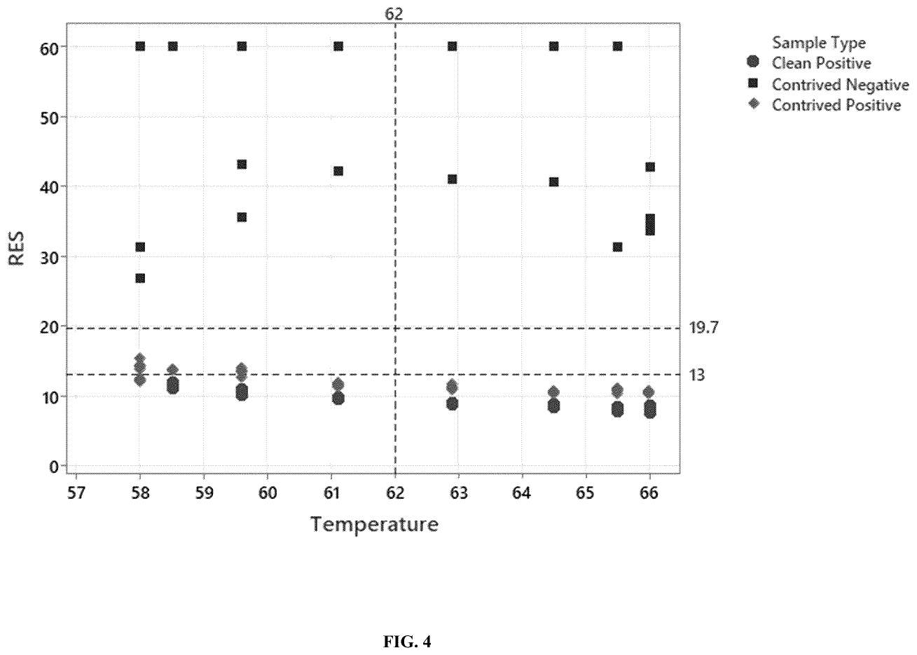

Compounds were added to the LAMP reaction mixture containing target-specific primers, dNTPs, DNA polymerase, reverse transcriptase, and the target RNA sequence. This reaction mixture was incubated at 62° C. (or the specified temperature (FIG. 4)) for at least 50 minutes and the increase in fluorescence was assessed to determine the time to positivity (Tp). Data shown in FIGS. 1A-B support that these compounds are compatible with LAMP assays at concentrations up to 16 μM.

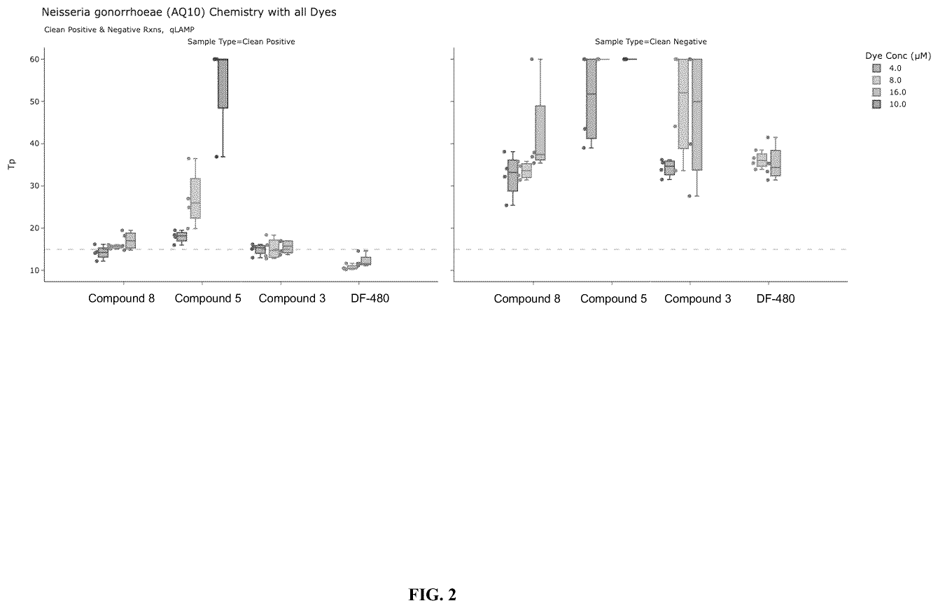

Performance of LAMP assays in FIG. 2 shows that results can be impacted by the choice of compound. For the Neisseria gonorrhoeae (AQ10), there is detrimental impact on Tp observed with Compound 5 at ≥8 μM where a delay in Tp is observed.

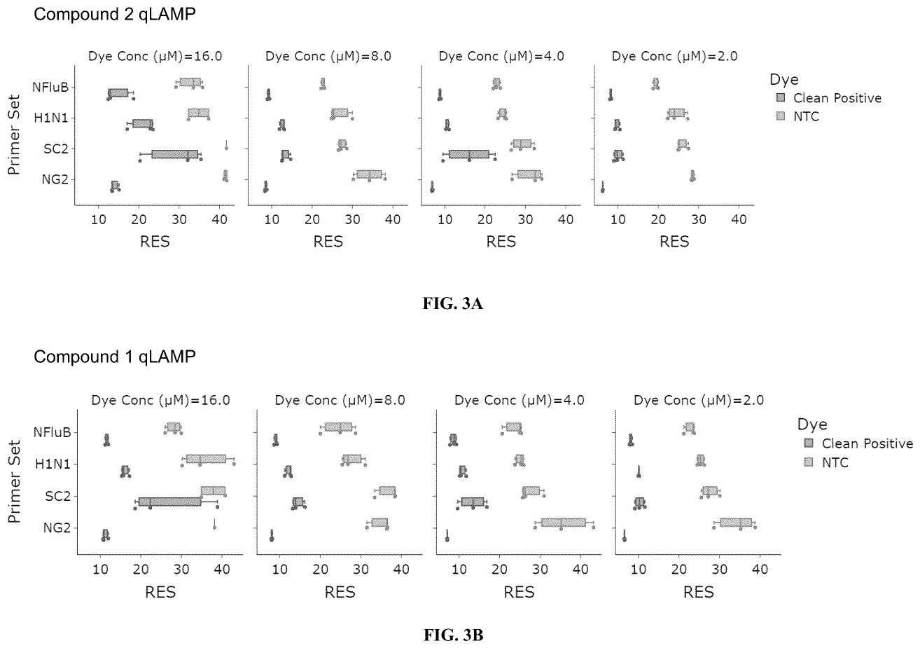

Furthermore, FIGS. 3A-B show that addition of quaternary ammonium group is inhibitory to LAMP at >2 μM concentrations. Examples in FIGS. 3A-B are for Flu B, H1N1, SC2, and NG2 assay chemistries under clean conditions.

FIG. 4 shows optimal temperature analysis for Compound 3 with for the Neisseria gonorrhoeae (AQ10) assay chemistry. Data shown is under clean and eluted vaginal swabs (contrived conditions). Good performance is observed at ≥61° C. There is detrimental impact on performance at <60° C. (delay in Tp).

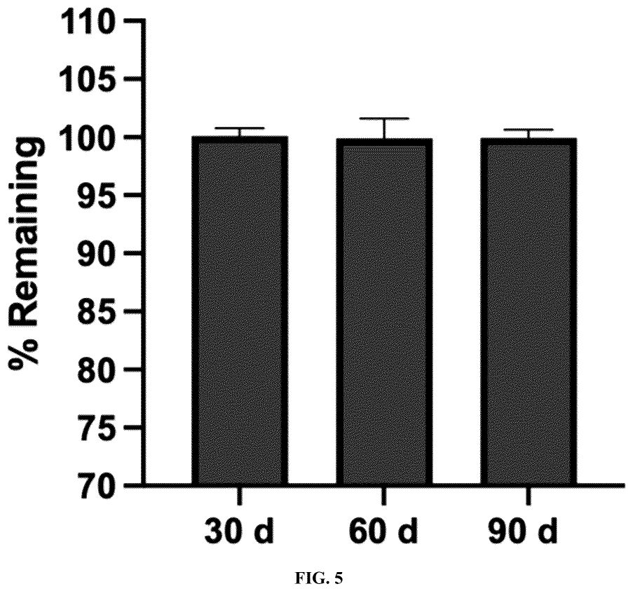

It was also determined that Compound 3 is stable under accelerated stability conditions from lyophilized chemistry (FIG. 5). As assayed by HPLC, Compound 3 is stable under these conditions at the 3-month time point (98.7±0.33% remaining), corresponding to 6 months stability at room temperature (15-30° C.).

### Example 3. Absorbance/Emission Assays

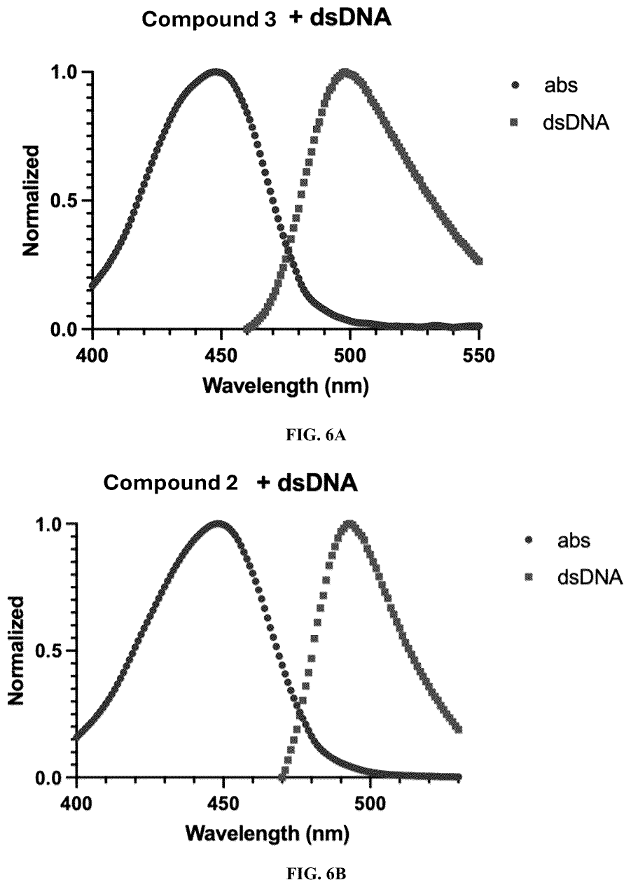

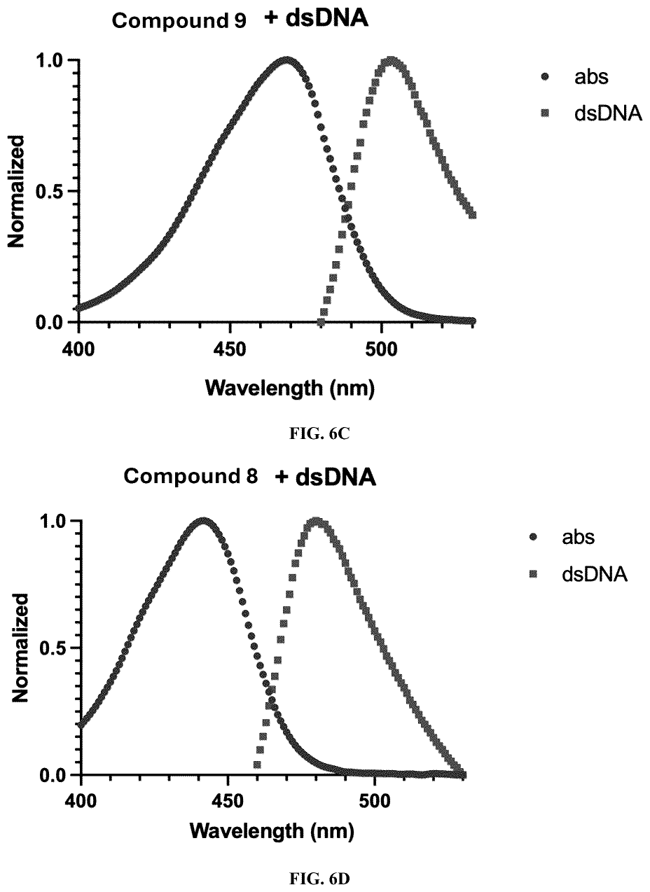

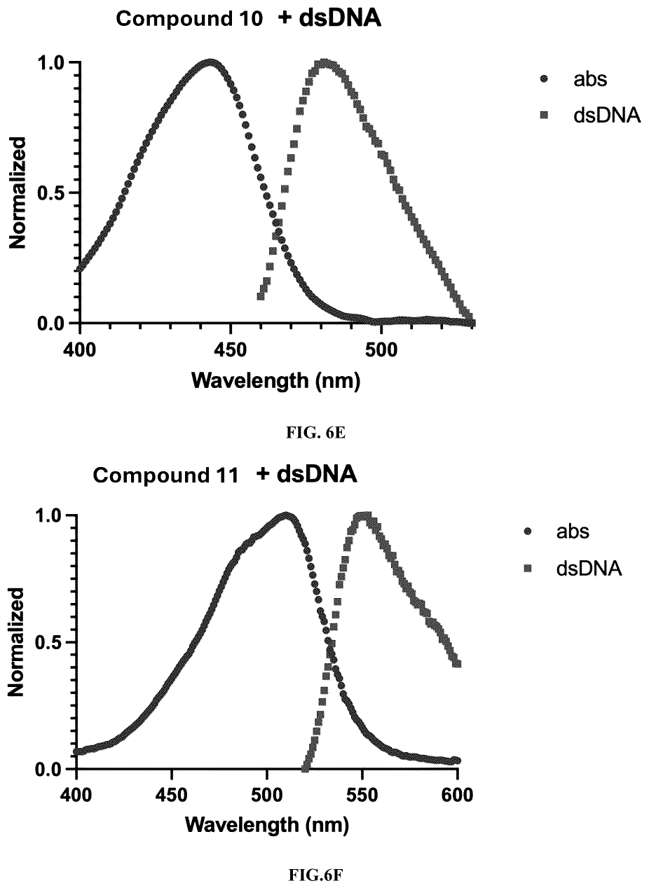

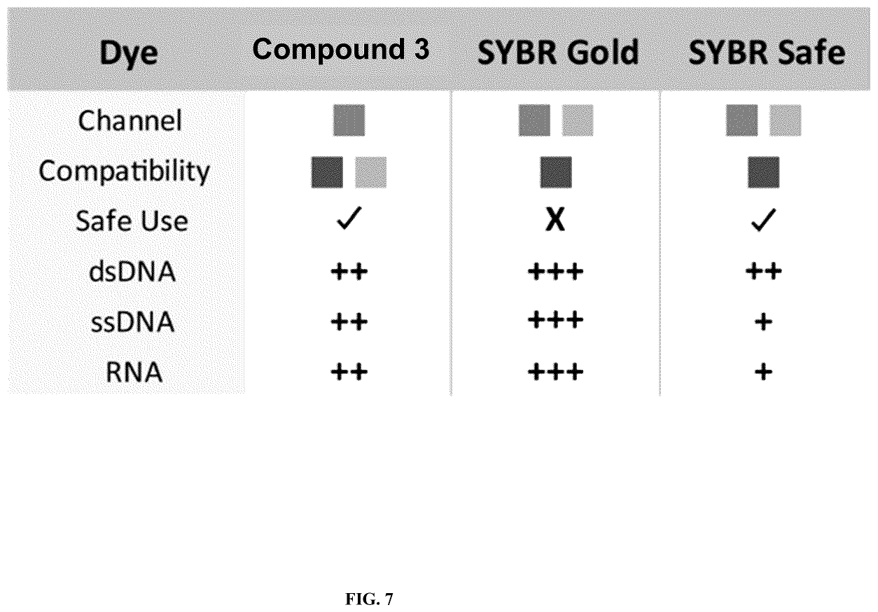

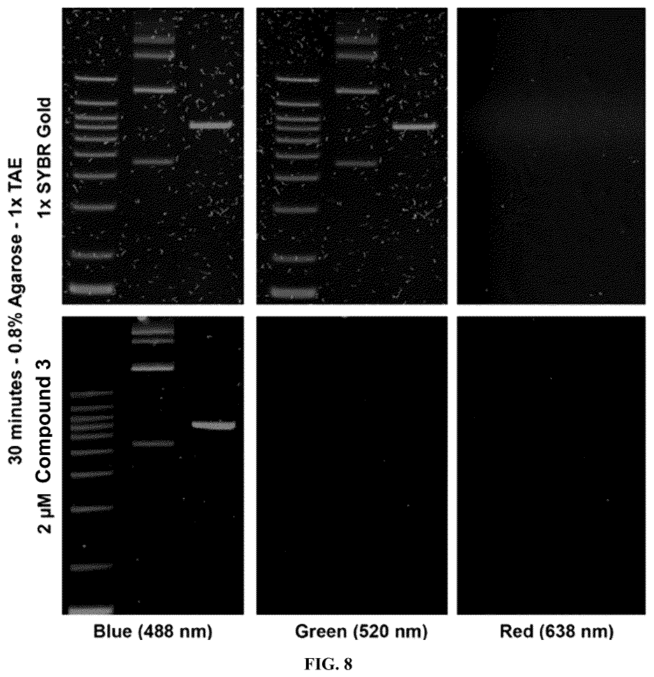

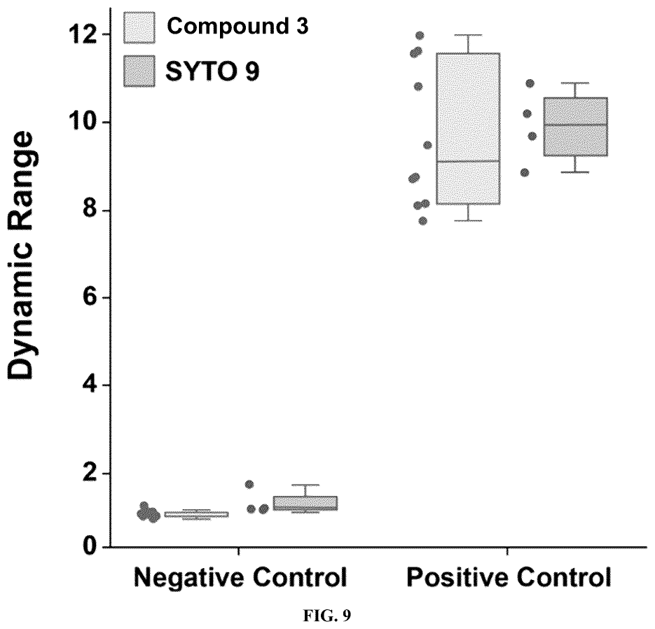

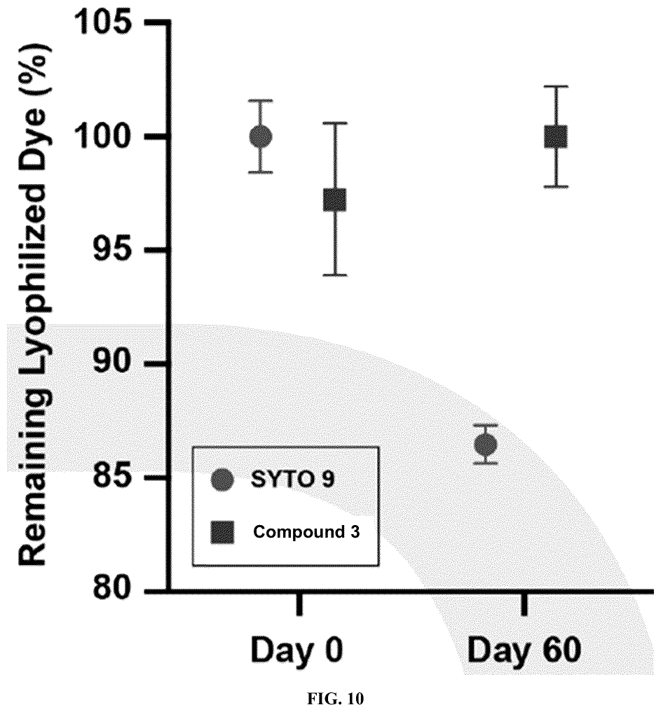

Fluorescence readings (FIGS. 6A-F) were performed using 10 mm path length, 700 μL quartz cuvettes on Horiba Fluorolog-Q fluorometer instrument. All measurements were taken at elevated temperature (62±2° C.) in pH 8.8 isoamplification buffer containing 20 mM Tris-HCl. For each measurement, 2 μM of dye was combined with 2 μM of hairpin DNA. Absorption spectra were recorded at ambient temperature (25±2° C.) on a DeNovix DS-11 spectrophotometer instrument. Normalized spectra are shown for clarity. The benzothiazole dye compounds have large Stokes shift >40 nm, which allows a substantial portion of the output fluorescence to pass through the filter covering the photodiode detection component of the device. Furthermore, this large Stokes shift allows for multicomponent imaging (i.e., using multiple dyes that do not have overlapping spectral windows (FIG. 8)).

### Example 4. DNA Quantification

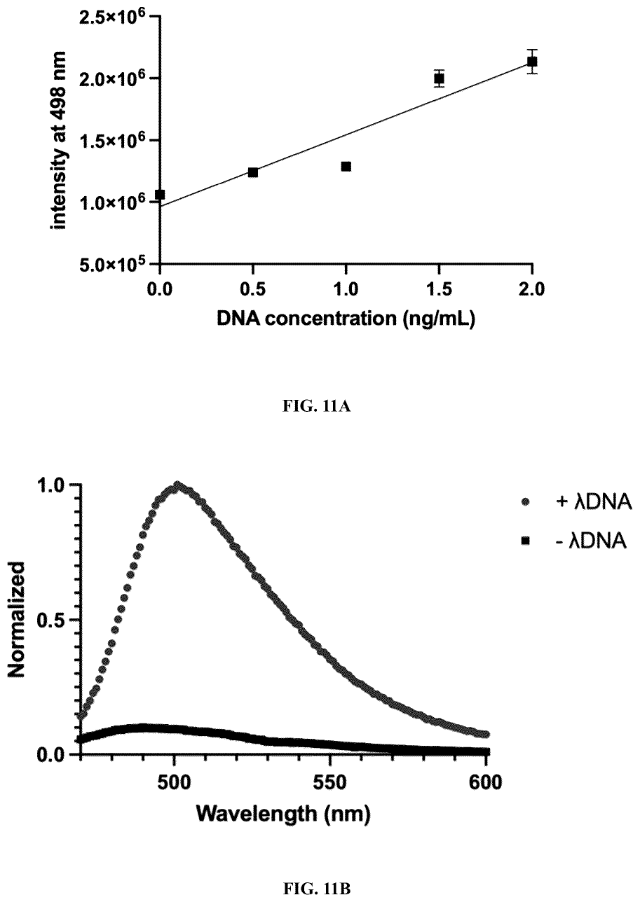

Fluorescence readings (FIGS. 11A-B) were performed using 10 mm path length, 700 μL quartz cuvettes on Horiba Fluorolog-Q fluorometer instrument. All measurements were taken at ambient temperatures (25±2° C.) in pH 8.8 isoamplification buffer containing 20 mM Tris-HCl. For each measurement, 10 μM of dye was combined with the specified λDNA concentration.

### EMBODIMENTS

Embodiment 1. A compound of formula (I):

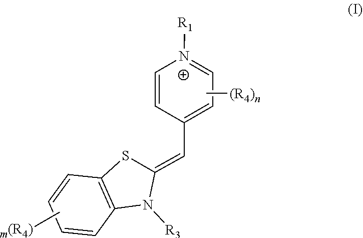

or a salt thereof, wherein:
n is 1-4
m is 0-4
R₁ is substituted or unsubstituted aliphatic, substituted or unsubstituted heteroaliphatic, substituted or unsubstituted aryl, or substituted or unsubstituted heteroaryl;
R₂ is, independently for each occurrence, halogen, —OR₅, —SR₅, —N(R₅)₂, substituted or unsubstituted aliphatic, substituted or unsubstituted heteroaliphatic, substituted or unsubstituted carbocyclyl, substituted or unsubstituted heterocyclyl, substituted or unsubstituted aryl, or substituted or unsubstituted heteroaryl; wherein two adjacent R₂ groups may combine to form a saturated, fused ring;
R₃ is substituted or unsubstituted aliphatic;
R₄ is, independently for each occurrence, halogen, —NO₂, —OR₆, —SR₆, —N(R₆)₂, —substituted or unsubstituted aliphatic, substituted or unsubstituted heteroaliphatic, substituted or unsubstituted carbocyclyl, substituted or unsubstituted heterocyclyl, substituted or unsubstituted aryl, or substituted or unsubstituted heteroaryl;
R₅ is, independently for each occurrence, hydrogen, or substituted or unsubstituted aliphatic; and
R₆ is, independently for each occurrence, hydrogen, or substituted or unsubstituted aliphatic.

Embodiment 2. The compound of embodiment 1, of formula (Ia):

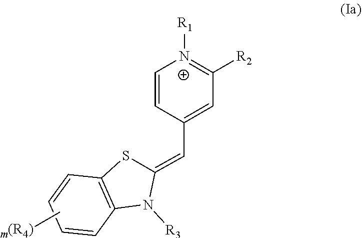

or a salt thereof.

Embodiment 2. The compound of embodiment 1, of formula (Ib):

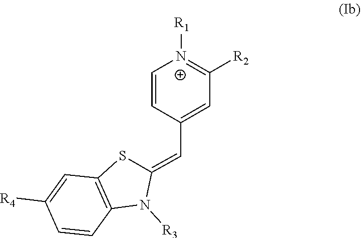

or a salt thereof.

Embodiment 3. The compound or salt of embodiment 1 or 2, wherein R₁ is substituted or unsubstituted alkyl.

Embodiment 4. The compound or salt of embodiment 3, wherein R₁ is substituted with a quaternary amine (e.g., trimethylammonium).

Embodiment 5. The compound or salt of any one of embodiments 1-4, wherein at least one R₂ is substituted or unsubstituted heterocyclyl, substituted or unsubstituted aryl, or substituted or unsubstituted heteroaryl.

Embodiment 6. The compound or salt of embodiment 5, wherein at least one R₂ is selected from:

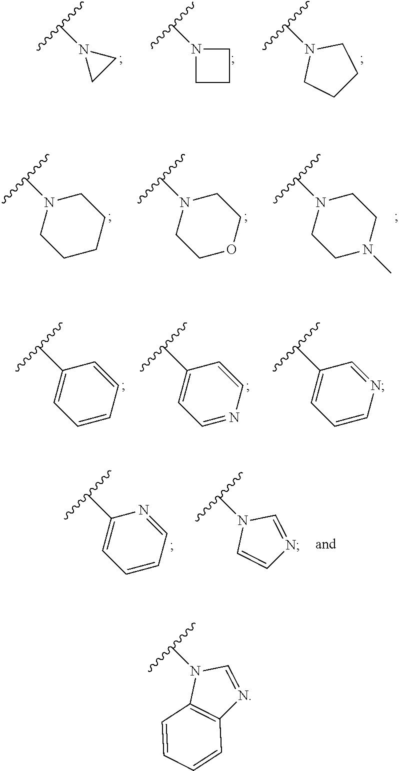

Embodiment 7. The compound or salt of any one of embodiments 1-6, R₃ is substituted or unsubstituted alkyl.

Embodiment 8. The compound or salt of embodiment 7, wherein R₃ is substituted with a quaternary amine (e.g., trimethylammonium).

Embodiment 9. The compound or salt of any one of embodiments 1-8, wherein R₄ is absent, or is —OR₆.

Embodiment 10. The compound or salt of embodiment 9, wherein R₄ is —OR₆.

Embodiment 11. The compound or salt of any one of embodiments 1-10, wherein R₆ is substituted or unsubstituted alkyl.

Embodiment 12. The compound or salt of embodiment 11, wherein R₆ is substituted with a quaternary amine (e.g., trimethylammonium).

Embodiment 13. The compound or salt of embodiment 9, wherein R₄ is absent.

Embodiment 14. The compound of embodiment 1, selected from the group consisting of

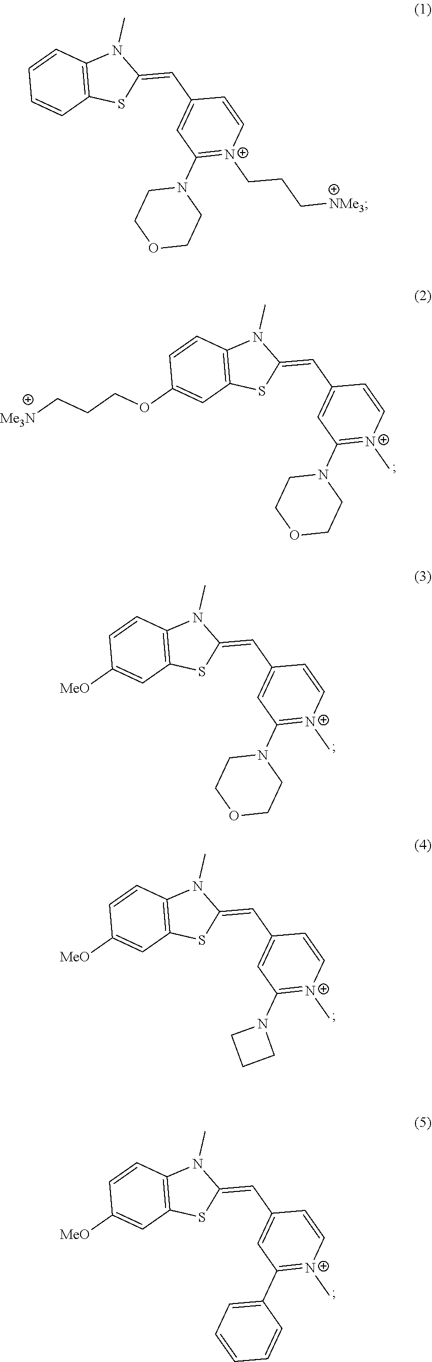

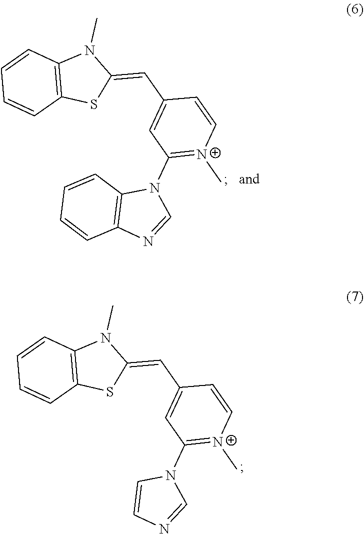 [SMILES: CN1/C(=C/c2cc[n+](C)c(-n3cnc4ccccc43)c2)Sc2ccccc21 ; CN1/C(=C/c2cc[n+](C)c(-n3ccnc3)c2)Sc2ccccc21]

or a salt thereof.

Embodiment 15. A composition comprising a compound of any one of embodiments 1-14.

Embodiment 16. The composition of embodiment 15, wherein the composition is a solution or a suspension.

Embodiment 17. The composition of embodiment 24, wherein the concentration of the compound is in the range of 0.001-100 μM.

Embodiment 18. The composition of any one of embodiments 15-17, wherein the composition further comprises a test sample.

Embodiment 19. The composition of embodiment 18, wherein the test sample is of an animal bodily tissue or fluid.

Embodiment 20. The composition of embodiment 19, wherein the animal is a human.

Embodiment 21. The composition of any one of embodiments 15-20, wherein the test sample comprises an analyte.

Embodiment 22. The composition of embodiment 21, wherein the analyte is a nucleic acid.

Embodiment 23. The composition of embodiment 22, wherein the nucleic acid is DNA.

Embodiment 24. The composition of embodiment 23, wherein the DNA is double-stranded DNA.

Embodiment 25. The composition of any one of embodiments 22-24, wherein the nucleic acid is of a virus or bacterium.

Embodiment 26. The composition of embodiment 25, wherein the virus is SARS-CoV-2.

Embodiment 27. A method of detecting the presence or absence of an analyte in a test sample, comprising contacting the test sample with a compound of any one of embodiments 1-14 in the presence of an energy (E1) and detecting emission of an energy (E2) from the sample.

Embodiment 28. The method of embodiment 27, wherein the test sample is of an animal bodily tissue or fluid.

Embodiment 29. The method of embodiment 28, wherein the animal is a human.

Embodiment 30. The method of any one of embodiments 27-29, wherein the analyte is a nucleic acid.

Embodiment 31. The method of embodiment 30, wherein the nucleic acid is DNA.

Embodiment 32. The method of embodiment 31, wherein the DNA is double-stranded DNA.

Embodiment 33. The method of any one of embodiments 30-32, wherein the nucleic acid is of a virus or bacterium.

Embodiment 34. The method of embodiment 33, wherein the virus is SARS-CoV-2.

Embodiment 35. The method of any one of embodiments 27-34, wherein E1 is electromagnetic radiation having a wavelength of about 400 nm to about 800 nm.

Embodiment 36. The method of any one of embodiments 27-35, wherein E2 is emitted by fluorescence of the compound.

Embodiment 37. The method of embodiment 36, wherein E2 of the test sample is lower when the analyte is present.

Embodiment 38. The method of embodiment 36, wherein E2 of the test sample is higher when the analyte is present.

Embodiment 39. The method of any one of embodiments 27-38, wherein the test sample is a solution or suspension.

Embodiment 40. The method of embodiment 39, wherein the compound is present in an amount of 1-10 nmol per milliliter of test sample.

1. A compound of Formula (I): 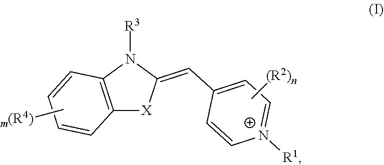
or a salt thereof, wherein:
n is 1-4;
m is 0-4;
X is oxygen or sulfur;
R¹ is hydrogen, substituted or unsubstituted alkyl, substituted or unsubstituted heteroalkyl, or substituted or unsubstituted heteroaryl;
R² is, independently for each occurrence, halo, —OR⁵, —SR⁵, —N(R⁵)₂, substituted or unsubstituted alkyl, substituted or unsubstituted heteroalkyl, substituted or unsubstituted carbocyclyl, substituted or unsubstituted heterocyclyl, substituted or unsubstituted aryl, or substituted or unsubstituted heteroaryl; wherein two adjacent R² groups may combine to form a fused substituted or unsubstituted aryl, a fused substituted or unsubstituted heteroaryl, a fused substituted or unsubstituted carbocyclyl, or a fused substituted or unsubstituted heterocyclyl;
R³ is substituted or unsubstituted alkyl;
R⁴ is, independently for each occurrence, halo, —NO₂, —OR⁶, —SR⁶, —N(R⁶)₂, substituted or unsubstituted alkyl, substituted or unsubstituted heteroalkyl, substituted or unsubstituted carbocyclyl, substituted or unsubstituted heterocyclyl, substituted or unsubstituted aryl, or substituted or unsubstituted heteroaryl;
R⁵ is, independently for each occurrence, hydrogen, substituted or unsubstituted alkyl, nitrogen protecting group when attached to a nitrogen, oxygen protecting group when attached to an oxygen, or a sulfur protecting group when attached to a sulfur, or when R² is —N(R⁵)₂, two instances of R⁵ may be joined to form a substituted or unsubstituted heterocyclyl or a substituted or unsubstituted heteroaryl; and
R⁶ is, independently for each occurrence, hydrogen, substituted or unsubstituted alkyl, nitrogen protecting group when attached to a nitrogen, oxygen protecting group when attached to an oxygen, or a sulfur protecting group when attached to a sulfur.

2. The compound of claim 1, or a salt thereof, wherein X is sulfur.

3. The compound of claim 1, or a salt thereof, wherein R¹ is hydrogen, substituted or unsubstituted alkyl, or substituted or unsubstituted heteroalkyl.

4-6. (canceled)

7. The compound of claim 3, or a salt thereof, wherein R¹ is unsubstituted alkyl.

8. The compound of claim 7, of a salt thereof, wherein R¹ is methyl.

9-10. (canceled)

11. The compound of claim 1, or a salt thereof, wherein n is 1 or 2.

12. The compound of claim 1, wherein the compound is of the Formula (I-a) or (I-b): 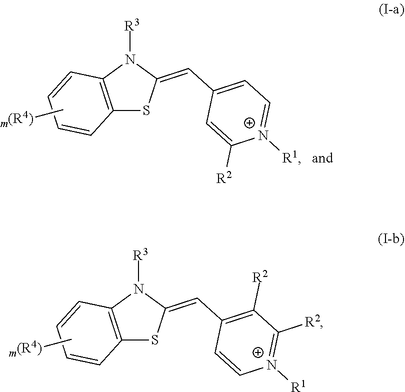
or a salt thereof.

13. The compound of claim 1, or a salt thereof, wherein at least one instance of R² is substituted or unsubstituted heterocyclyl, substituted or unsubstituted aryl, substituted or unsubstituted heteroaryl, or —N(R⁵)₂, or two adjacent R² groups combine to form a fused substituted or unsubstituted aryl.

14. (canceled)

15. The compound of claim 1, or a salt thereof, wherein at least one instance of R² is selected from: 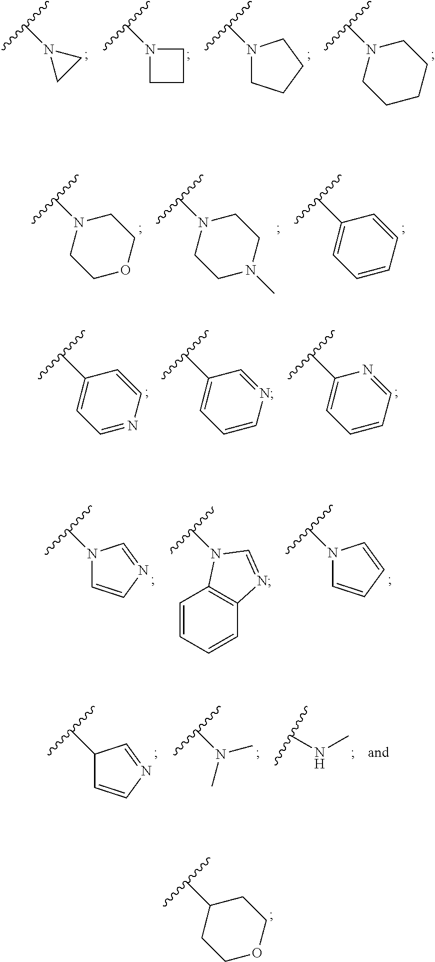
or two adjacent R² groups combine to form

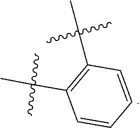 [SMILES: *c1ccccc1*]

16. (canceled)

17. The compound of claim 15, or a salt thereof, wherein at least one instance of R² is

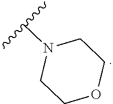 [SMILES: *N1CCOCC1]

18. The compound of claim 1, or a salt thereof, wherein R³ is unsubstituted alkyl.

19. The compound of claim 1, wherein the compound is of the Formula (I-c): 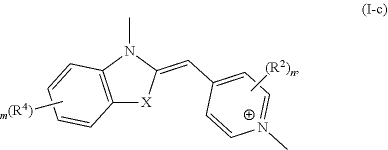
or a salt thereof.

20-25. (canceled)

26. The compound of claim 1, wherein the compound is of the Formula (I-e) or (I-f): 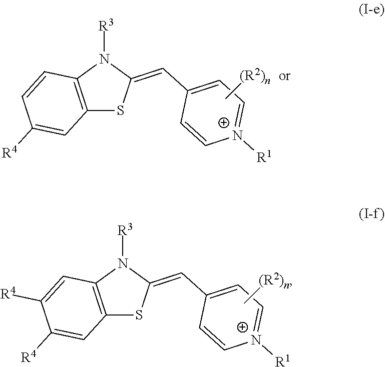
or a salt thereof.

27. (canceled)

28. The compound of claim 1, or a salt thereof, wherein at least one instance of R⁴ is substituted or unsubstituted alkyl, halo, or —OR⁶.

29. The compound of claim 28, or a salt thereof, wherein at least one instance of R⁴ is —OR⁶.

30-35. (canceled)

36. The compound of claim 1, selected from the group consisting of

| Compound Number | Structure |
|---|---|
| 1 | 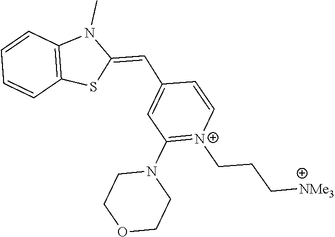 |
| 2 | 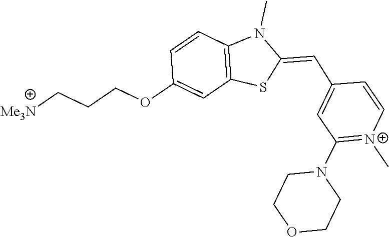 |
| 3 | 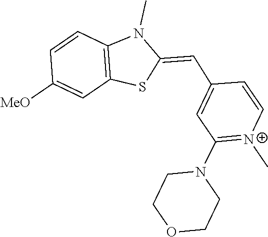 [SMILES: COc1ccc2c(c1)S/C(=C\c1cc[n+](C)c(N3CCOCC3)c1)N2C] |
| 4 | 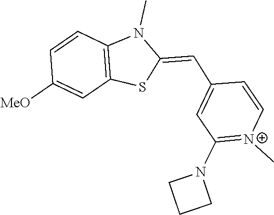 [SMILES: COc1ccc2c(c1)S/C(=C\c1cc[n+](C)c(N3CCC3)c1)N2C] |
| 5 | 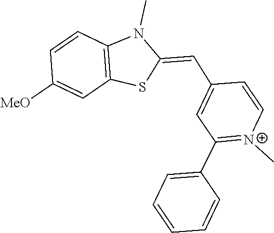 [SMILES: COc1ccc2c(c1)S/C(=C\c1cc[n+](C)c(-c3ccccc3)c1)N2C] |
| 6 | 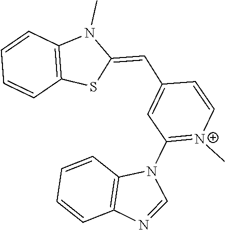 [SMILES: CN1/C(=C/c2cc[n+](C)c(-n3cnc4ccccc43)c2)Sc2ccccc21] |
| 7 | 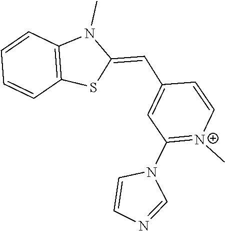 [SMILES: CN1/C(=C/c2cc[n+](C)c(-n3ccnc3)c2)Sc2ccccc21] |
| 8 | 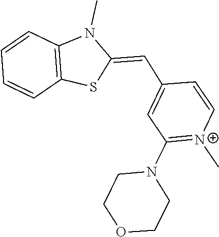 [SMILES: CN1/C(=C/c2cc[n+](C)c(N3CCOCC3)c2)Sc2ccccc21] |
| 9 | 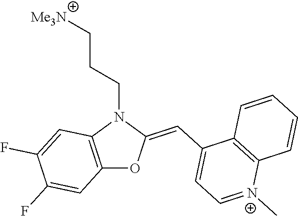 |
| 10 | 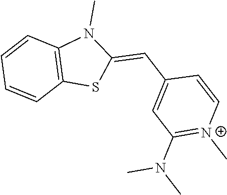 [SMILES: CN(C)c1cc(/C=C2\Sc3ccccc3N2C)cc[n+]1C] |
| 11 | 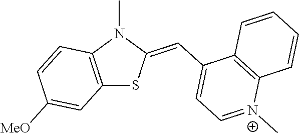 [SMILES: COc1ccc2c(c1)S/C(=C\c1cc[n+](C)c3ccccc13)N2C] |

or a salt thereof.

37-43. (canceled)

44. A composition comprising a compound of Formula (I): 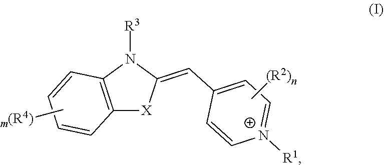
or a salt thereof, wherein:
n is 1-4;
m is 0-4;
X is oxygen or sulfur;
R¹ is hydrogen, substituted or unsubstituted alkyl, substituted or unsubstituted heteroalkyl, or substituted or unsubstituted heteroaryl;
R² is, independently for each occurrence, halo, —OR⁵, —SR⁵, —N(R⁵)₂, substituted or unsubstituted alkyl, substituted or unsubstituted heteroalkyl, substituted or unsubstituted carbocyclyl, substituted or unsubstituted heterocyclyl, substituted or unsubstituted aryl, or substituted or unsubstituted heteroaryl; wherein two adjacent R² groups may combine to form a fused substituted or unsubstituted aryl, a fused substituted or unsubstituted heteroaryl, a fused substituted or unsubstituted carbocyclyl, or a fused substituted or unsubstituted heterocyclyl;
R³ is substituted or unsubstituted alkyl;
R⁴ is, independently for each occurrence, halo, —NO₂, —OR⁶, —SR⁶, —N(R⁶)₂, substituted or unsubstituted alkyl, substituted or unsubstituted heteroalkyl, substituted or unsubstituted carbocyclyl, substituted or unsubstituted heterocyclyl, substituted or unsubstituted aryl, or substituted or unsubstituted heteroaryl;
R⁵ is, independently for each occurrence, hydrogen, substituted or unsubstituted alkyl, nitrogen protecting group when attached to a nitrogen, oxygen protecting group when attached to an oxygen, or a sulfur protecting group when attached to a sulfur, or when R² is —N(R⁵)₂, two instances of R⁵ may be joined to form a substituted or unsubstituted heterocyclyl or a substituted or unsubstituted heteroaryl; and
R⁶ is, independently for each occurrence, hydrogen, substituted or unsubstituted alkyl, nitrogen protecting group when attached to a nitrogen, oxygen protecting group when attached to an oxygen, or a sulfur protecting group when attached to a sulfur.

45-47. (canceled)

48. The composition of claim 44, wherein the composition further comprises a test sample.

49-62. (canceled)

63. A method of detecting the presence or absence of an analyte in a test sample, comprising contacting the test sample with a compound of Formula (I): 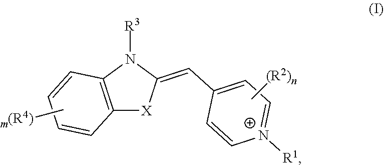
or a salt thereof, in the presence of an energy (E1) and detecting emission of an energy (E2) from the sample, wherein:
n is 1-4;
m is 0-4;
X is oxygen or sulfur;
R¹ is hydrogen, substituted or unsubstituted alkyl, substituted or unsubstituted heteroalkyl, or substituted or unsubstituted heteroaryl;
R² is, independently for each occurrence, halo, —OR⁵, —SR⁵, —N(R⁵)₂, substituted or unsubstituted alkyl, substituted or unsubstituted heteroalkyl, substituted or unsubstituted carbocyclyl, substituted or unsubstituted heterocyclyl, substituted or unsubstituted aryl, or substituted or unsubstituted heteroaryl; wherein two adjacent R² groups may combine to form a fused substituted or unsubstituted aryl, a fused substituted or unsubstituted heteroaryl, a fused substituted or unsubstituted carbocyclyl, or a fused substituted or unsubstituted heterocyclyl;
R³ is substituted or unsubstituted alkyl;
R⁴ is, independently for each occurrence, halo, —NO₂, —OR⁶, —SR⁶, —N(R⁶)₂, substituted or unsubstituted alkyl, substituted or unsubstituted heteroalkyl, substituted or unsubstituted carbocyclyl, substituted or unsubstituted heterocyclyl, substituted or unsubstituted aryl, or substituted or unsubstituted heteroaryl;
R⁵ is, independently for each occurrence, hydrogen, substituted or unsubstituted alkyl, nitrogen protecting group when attached to a nitrogen, oxygen protecting group when attached to an oxygen, or a sulfur protecting group when attached to a sulfur, or when R² is —N(R⁵)₂, two instances of R⁵ may be joined to form a substituted or unsubstituted heterocyclyl or a substituted or unsubstituted heteroaryl; and
R⁶ is, independently for each occurrence, hydrogen, substituted or unsubstituted alkyl, nitrogen protecting group when attached to a nitrogen, oxygen protecting group when attached to an oxygen, or a sulfur protecting group when attached to a sulfur.

64-82. (canceled)

83. A kit comprising:
a compound of claim 1, or a salt thereof; and instructions for use.
